(19) United States
(12) Patent Application Publication
(10) Pub. No.: US 2025/0177315 A1
(43) Pub. Date: Jun. 5, 2025

(54) PRE-FUNCTIONALIZED HARD SHELL CAPSULE WITH ACCELERATED DRUG RELEASE AT PH VALUE 5 TO 5.5

(71) Applicant: Evonik Operations GmbH, Essen (DE)

(72) Inventors: Hans Bär, Michelstadt (DE); Priyanka Haksar, Thane (West) (IN); Nilam Bharambe, Dombivali West (IN); Shraddha Joshi, Thane (IN)

(73) Assignee: Evonik Operations GmbH, Essen (DE)

(21) Appl. No.: 18/846,094

(22) PCT Filed: Mar. 6, 2023
(86) PCT No.: PCT/EP2023/055524

§ 371 Date: Sep. 11, 2024

(30) Foreign Application Priority Data
Mar. 15, 2022 (IN) 202211014107
Apr. 27, 2022 (EP) 22170267.3

(51) Int. Cl. A61K 9/48

(52) U.S. Cl. CPC A61K 9/4891; A61K 9/4866

(57) ABSTRACT

A process prepares a polymer-coated hard shell capsule including an intermediate coating layer, an enteric coating layer, and optionally a top coating layer, suitable as a container for pharmaceutical or nutraceutical biologically active ingredients. The hard shell capsule includes a body and a cap, and in a closed state, the cap overlaps the body either in a pre-locked state or in a final-locked state. The hard shell capsule is provided in the pre-locked state and is coated with a first coating solution, suspension or dispersion to obtain the intermediate coating layer of the hard shell capsule in the pre-locked state. Thereafter, the hard shell capsule is coated with a second coating solution, suspension or dispersion, which is different from the first coating solution, suspension or dispersion, to obtain the enteric coating layer of the hard shell capsule in the pre-locked state.

### FIELD OF THE INVENTION

The invention refers to a process for preparing a polymer-coated hard shell capsule comprising at least an intermediate coating layer and an enteric coating layer and optionally a top coating layer, suitable as container for pharmaceutical or nutraceutical biologically active ingredients, wherein the hard shell capsule comprises a body and a cap, wherein in the closed state the cap overlaps the body either in a pre-locked state or in a final-locked state, wherein the hard shell capsule is provided in the pre-locked state and is coated
with a first coating solution, suspension or dispersion comprising or consisting of
a1) at least one polymer;
b1) at least one alkaline agent;
c1) optionally at least one glidant; and
d1) optionally at least one additive, different from a1) to c1);
to obtain the intermediate coating layer of the hard shell capsule in the pre-locked state; and thereafter
is coated with a second coating solution, suspension or dispersion, which is different from the first coating solution, suspension or dispersion, comprising or consisting of
a2) at least two polymers;
b2) optionally at least one glidant;
c2) optionally at least one emulsifier;
d2) optionally at least one plasticizer;
e2) optionally at least one biologically active ingredient; and
f2) optionally at least one additive, different from a2) to e2);
wherein the first polymer is an anionic polymer having a T_{gm}≥35° C.;
wherein the second polymer is a polymer having a T_{gm} of ≤30° C.;
to obtain the enteric coating layer of the hard shell capsule in the pre-locked state, wherein the total coating amount of the coating layers is 2.0 to 10 mg/cm². Furthermore, the invention refers to a polymer-coated hard shell capsule obtained from the process according to the invention and the use of the polymer-coated hard shell capsule for providing less than 10% drug release at 0.1N HCl for 120 minutes followed by at least 80% drug release at pH value 5 within additional 150 min.

### BACKGROUND

A favorable location for release and absorption of biologically active ingredients in the gastro-intestinal tract is the duodenum. In order to achieve targeted release of the biologically active ingredients they can be provided as pellets or within capsules having a specific coating of one or more coating layers, which needs to be adapted to the target, pellet or capsule and the requirements of the biologically active ingredient.

A release of biologically active ingredient in the duodenum is important for the treatment of e.g., duodenal ulcer but also systemic drug absorption as for example the mucus layer composition changes and thickness (see below) increases, thus early absorption of low bioavailable drugs benefit from early release in the duodenum.

Object of the present invention was to provide a system for pre-coated capsules showing targeted release in the duodenum, i.e., at a pH value of about 5, preferably suitable for automatic capsule filling.

In the prior art coating systems, which are suitable for a release at a pH value of about 5 are for example described in WO 2021115648 A1. The document discloses that the proposed coating systems are useful for capsules in general. However, examples and detailed disclosure is only given for pellets. In view of this reference, it has been found that the explicitly disclosed coating used in the examples is not suitable for use with pre-coated capsules, thus this reference teaches away from the present invention.

The inventors of the present invention have surprisingly found that for pre-coated capsules the specific intermediate coating layer and enteric coating layer are required to solve the above-mentioned problem.

### SUMMARY OF THE INVENTION

In a first aspect the invention refers to a process for preparing a polymer-coated hard shell capsule comprising at least an intermediate coating layer and an enteric coating layer and optionally a top coating layer, suitable as container for pharmaceutical or nutraceutical biologically active ingredients, wherein the hard shell capsule comprises a body and a cap, wherein in the closed state the cap overlaps the body either in a pre-locked state or in a final-locked state, wherein the hard shell capsule is provided in the pre-locked state and is coated with a first coating solution, suspension or dispersion comprising or consisting of
a1) at least one polymer;
b1) at least one alkaline agent;
c1) optionally at least one glidant; and
d1) optionally at least one additive, different from a1) to c1);
to obtain the intermediate coating layer of the hard shell capsule in the pre-locked state; and thereafter
is coated with a second coating solution, suspension or dispersion, which is different from the first coating solution, suspension or dispersion, comprising or consisting of
a2) at least two polymers;
b2) optionally at least one glidant;
c2) optionally at least one emulsifier;
d2) optionally at least one plasticizer;
e2) optionally at least one biologically active ingredient; and
f2) optionally at least one additive, different from a2) to e2);
wherein the first polymer is an anionic polymer having a T_{gm}≥35° C., preferably 35 to 155° C., more preferably 80 to 145° C., most preferably 90 to 125° C.;
wherein the second polymer is a polymer having a T_{gm} of ≤30° C., preferably ≤15° C.; to obtain the enteric coating layer of the hard shell capsule in the pre-locked state, wherein the total coating amount of the coating layers is 2.0 to 10 mg/cm².

In a second aspect the invention refers to a polymer-coated hard shell capsule obtained from the process according to the present invention.

In a third aspect the invention refers to the use of the polymer-coated hard shell capsule according to the present invention for delayed release, i.e. for providing less than 10% drug release at 0.1N HCl for 120 minutes followed by at least 80% drug release at pH value 5 within additional 150 min.

### DETAILED DESCRIPTION OF THE INVENTION

### Hard Shell Capsules

Hard shell capsules for pharmaceutical or nutraceutical purposes are well known to a skilled person. A hard shell capsule is a two-piece encapsulation capsule comprising of the two capsule halves, called the body and the cap. The capsule body and cap material is usually made from a hard and sometimes brittle material. The hard shell capsule comprises a body and a cap. Body and cap are usually of a one end open cylindrical form with closed rounded hemispherical ends on the opposite end. The shape and size of the cap and body are such that the body can be pushed telescopically with its open end into the open end of the cap.

The body and the cap comprise a potential overlapping, matching area (overlap area) outside the body and inside the cap which partially overlap when the capsule is closed in the pre-locked state and totally overlap in the final-locked state. When the cap is partially slid over the overlapping matching area of the body then the capsule is in the pre-locked state. When the cap is totally slid over the overlapping matching area of the body then the capsule is in the final-locked state. The maintenance of the pre-locked state or of the final-locked state is usually supported by snap-in locking mechanisms of the body and the cap such as matching encircling notches or dimples, preferably elongated dimples.

Usually, the body is longer than the cap. The outside overlapping area of the body can be covered by the cap in order to close or to lock the capsule. In the closed state the cap covers the outside overlap area of the body either in a pre-locked state or in a final-locked state. In the final-locked state the cap covers the outside overlap area of the body in total, in the pre-locked state the cap overlaps the outside overlapping area of the body only partially. The cap can be slid over the body to be fixed in usually one of two different positions in which the capsule is closed either in a pre-locked state or in a final-locked state.

Hard shell capsules are commercially available in different sizes. Hard shell capsules are usually delivered as empty containers with the body and cap already positioned in the pre-locked state and on demand as separate capsules halves, bodies and caps. The pre-locked hard shell capsules can be provided to a capsule-filling machine, which performs the opening, filling and closing of the capsule into the final-locked state. Usually, hard shell capsules are filled with dry materials, for instance with powders or granules or pellets or mini tablets or sub-micron particles, or viscous liquids comprising a biologically active ingredient.

The cap and body are provided with closure means that are advantageous for the pre-locking (temporary) and/or final locking of the capsule. Therefore, elevated points can be provided on the inner wall of the cap and somewhat larger indented points are provided on the outer wall of the body, which are arranged so that when the capsule is closed the elevations fit into the indentations. Alternatively, the elevations can be formed on the outer wall of the body and the indentations on the inner wall of the cap. Arrangements in which the elevations or indentations arranged in a ring or spiral around the wall. Instead of the point-like configuration of the elevations and indentations, these may encircle the wall of the cap or body in an annular configuration, although advantageously recesses and openings are provided which enable an exchange of gases into and out of the capsule interior. One or more elevations can be provided in an annular arrangement around the inner wall of the cap and the outer wall of the body such that, in the final-locked position of the capsule, an elevation on the cap is located adjacent to an elevation on the body. Sometimes elevations are formed on the outside of the body close to the open end and indentations are formed in the cap close to the open end such that the elevations on the body latch into the indentations in the cap in the final-locked position of the capsule. The elevations can be such that the cap can be opened in the pre-locked state at any time without damage to the capsule or, alternatively, so that once it has been closed the capsule cannot be opened again without destroying it. Capsules with one or more such latching mechanisms (latches) (for example two encircling grooves) are preferred. More preferred are capsules with at least two such latching means which secure the two capsule parts to different degrees. In a part of this kind, a first latching (dimples or encircling notches) means can be formed close to the openings in the capsule cap and the capsule body and a second latching (encircling notches) can be shifted somewhat further towards the closed end of the capsule parts. The first latching means secures the two capsule parts less strongly than the second does. This variant has the advantage that after the production of the empty capsules the capsule cap and capsule body can initially be pre-locked joined together using the first latching mechanism. In order to fill the capsule the two capsule parts are then separated again. After filling, the two capsule parts are pushed together until the second set of latches firmly secures the capsule parts in a final-locked state.

Preferably, the body and the cap of the hard shell capsule are comprising each encircling notches and/or dimples in the area, where the cap can be slid over the body. Encircling notches of the body and dimples of the cap match to each other to provide a snap-in or snap into-place mechanism. The dimples can be circular or elongated (oval) in the longitudinal direction. Encircling notches of the body and encircling notches of the cap (closely matched rings) also match to each other to provide a snap-in or snap into-place mechanism. This allows the capsule to be closed by a snap-into-place mechanism either in a pre-locked state or in a final-locked state.

Preferably, matching encircling notches of the body and elongated dimples of the cap are used to fix the body and the cap to each other in the pre-locked state. Matching encircling notches of the body and the cap are preferably used to fix or lock the body and the cap to each other in the final-locked state.

The area, where the cap can be slid over the body can be called the overlapping area of the body and the cap or briefly the overlap area. If the cap overlaps the body only partially, maybe to 20 to 90 or 60 to 85% of the overlap area, the hard shell capsule is only partially closed (pre-locked). Preferably, in the presence of a locking mechanism, like matching encircling notches and/or dimples in body and cap, the partially closed capsule can be called pre-locked. When the capsule is polymer-coated in the pre-locked state the coating will cover the completely outer surface including that part of the overlap area of the body and cap that is not overlapped by the cap in this pre-locked state. When the capsule is polymer-coated in the pre-locked state and then closed to the final-locked state the coating of that part of the overlap area of the body and cap that was not overlapped by the cap in the pre-locked state will then become covered by the cap. The presence of that part of the coating which is then enclosed in the final-locked state between the body and the cap is sufficient for the hard shell capsule to be tightly sealed.

If the cap overlaps the body the total overlapping area of the body, the hard shell capsule is finally closed or in the final-locked state. Preferably, in the presence of a locking mechanism, like matching encircling notches and/or dimples in body and cap, the finally closed capsule can be called final-locked.

Usually, dimples are preferred for the fixing the body and the cap in the pre-locked state. As a non-binding rule the matching area of dimples is smaller than the matching area of encircling notches. Thus snapped-in dimples can be snapped-out again by applying less forces than those that would be necessary to snap-out a snapped-in fixation by matching encircling notches.

The dimples of the body and cap are located in the area, where the cap can be slid over the body match to each other in the pre-locked state by a snap in or snap into-place mechanism. There can be for example 2, 4, or preferably 6 notches or dimples located distributed circular around the cap.

Usually, the dimples of the cap are and the encircling notches of the body in the area, where the cap can be slid over the body match to each other so that they that allow the capsule to be closed by a snap-into-place mechanism in the pre-locked state. In the pre-locked state, the hard shell capsule can be re-opened manually or by a machine without damaging, because the forces needed to open are comparatively low. Thus, the “pre-locked state” is sometimes designated also as “loosely capped”.

Usually, the encircling notches or matching locking rings of the body and the cap in the area, where the cap can be slid over the body match to each other so that they allow the capsule to be closed by a snap-into-place mechanism in the final-locked state. In the final-locked state, the hard shell capsule cannot or can be only hardly be re-opened manually or by a machine without damaging, because the forces needed to open are comparatively high.

Usually dimples and the encircling notches are formed in the capsule body or capsule cap. When the capsule parts provided with these elevations and indentations are fitted into one another, ideally defined uniform gaps of from 10 microns to 150 microns, more particularly 20 microns to 100 microns, are formed along the contact surface between the capsule body and the capsule cap placed thereon.

Preferably, the body of the hard shell capsule comprises a tapered rim. The tapered rim prevents the rims of the body and the cap to collide and becoming damaged when the capsule is closed manually or by a machine.

In contrast to a hard shell capsule, a soft shell capsule is a welded one piece encapsulation capsule. A soft gel capsule is often made from blow molded soft gelling substances and is usually filled with liquids comprising a biologically active ingredient by injection. The invention is not concerned with welded soft shell one piece encapsulation capsules.

### Sizes of Hard Shell Capsules

A dosed, final-locked hard shell capsule can have a total length in the range from about 5 to 40 mm. The diameter of the cap can be in the range from about 1.3 to 12 mm. The diameter of the body can be in the range from about 1.2 to 11 mm. The length of the cap can be in the range from about 4 to 20 mm and that of the body in the range from 8 to 30 mm. The fill volume can be between about from 0.004 to 2 ml. The difference between the pre-locked length and the final-locked length can be about 1 to 5 mm.

Capsules can be divided into standardized sizes for example from sizes 000 to 5. A closed capsule of size 000 has, for example, a total length of about 28 mm with an outer diameter of the cap of about 9.9 mm and an outer diameter of the body of about 9.5 mm. The length of the cap is about 14 mm, that of the body about 22 mm. The fill volume is about 1.4 ml.

A closed capsule of size 5 has, for example, a total length of about 10 mm and an outer diameter of the cap of about 4.8 mm and an outer diameter of the body of about 4.6 mm. The length of the cap is about 5.6 mm, that of the body about 9.4 mm. The fill volume is about 0.13 ml.

A closed capsule of size 9 has, for example, a total length of about 8.4 mm and an outer diameter of the cap of about 2.7 mm. The fill volume is about 0.025 ml.

A size 0 capsule may show a length of about 23 to 24 mm in the pre-locked state and of about 20.5 to 21.5 mm in the final-locked state. Thus, the difference between the pre-locked length and the final-locked length can be about 2 to 3 mm.

### Coated Hard Shell Capsule

The invention is concerned with a polymer-coated hard shell capsule, obtained by the process as described herein.

The described process is especially useful for providing tightly closed polymer-coated hard shell capsules for pharmaceutical or nutraceutical dosage forms with gastric resistance and an intended rapid release in the duodenum.

### Material of the Body and the Cap

The base material of the body and the cap can be selected from hydroxypropyl methyl cellulose, starch, gelatin, pullulan and a copolymer of C1- to C4-alkylester of (meth)acrylic acid and (meth)acrylic acid. Preferred are hard shell capsules where body and cap are comprising or consisting of HPMC or gelatin, most preferred is HPMC because of its good adhesion properties for the polymer coating.

### Polymer or Polymer Mixture Comprised in the Intermediate or Enteric Coating Layer

In the following polymers, which are suitable for being used as the at least one polymer in the intermediate or one of the at least two polymers in the enteric coating layer are disclosed. The at least one polymer in the intermediate coating layer can be any of the below-mentioned polymers. The enteric coating layer requires a first polymer, wherein the first polymer is an anionic polymer having a T_{gm}≥35° C., preferably 35 to 155° C., more preferably 80 to 145° C., most preferably 90 to 125° C.; and a second polymer, wherein the second polymer is a polymer having a T_{gm} of ≤30° C., preferably ≤20° C., more preferred −10 to 20° C. Suitable anionic and neutral polymers are described in the following as well. If applicable to all polymers of both coating layers the expression “polymer comprised in/of the coating layer” or the like is used.

The polymer comprised in the coating layer is preferably a film-forming polymer.

The at least one polymer of the intermediate coating layer can be selected from the group of anionic polymers, cationic polymers and neutral polymers or any mixture thereof.

The selection of generic or specific polymer features or embodiments as disclosed herein can be combined without restriction with any other generic or specific selection of material or numerical features or embodiments as disclosed herein, such as capsule materials, capsule sizes, coating thicknesses, biologically active ingredients and any other features or embodiments as disclosed.

The coating layer, which can be a single layer or can comprise or consist of two or more individual layers, can comprise in total 10 to 100, 20 to 95, 30 to 90% by weight of one or more polymers, preferably (meth)acrylate copolymer(s).

The proportions of monomers mentioned for the respective polymers in general add up to 100% by weight.

The intermediate coating layer and the enteric coating layer are different from each other.

In a preferred embodiment a further coating layer, a top coating layer is contained. The top coating layer comprises at least one cationic polymer or at least one neutral polymer or any mxture thereof. In a preferred embodiment the top coating layer is selected from at least one natural polymer or a starch, preferably as described below or hydroxypropyl methylcellulose, most preferred is hydroxypropyl methylcellulose.

### Glass Transition Temperature T_{gm}

The glass transition temperature T_{gm} according to the present invention is preferably determined by Differential Scanning Calorimetry (DSC) according to ISO 11357-2:2013-05. The determination is performed with a heating rate of 20 K/min. The glass transition temperature T_{gm} can as well be determined by half step height method as described in section 10.1.2 of DIN EN ISO 11357-2.

### Anionic Polymers

The at least one polymer comprised in the coating layer can be an anionic polymer selected from the group of anionic (meth)acrylate copolymers, anionic polyvinyl polymers or copolymers and anionic celluloses.

### Anionic (Meth)Acrylate Copolymers

Preferably the anionic (meth)acrylate copolymer comprises 25 to 95, preferably 40 to 95, in particular 60 to 40, % by weight free-radical polymerized C1- to C12-alkyl esters, preferably C1- to C4-alkyl esters of acrylic or of methacrylic acid and 75 to 5, preferably 60 to 5, in particular 40 to 60% by weight (meth)acrylate monomers having an anionic group. The proportions mentioned in general add up to 100% by weight. However, it is also possible in addition, without this leading to an impairment or alteration of the essential properties, for small amounts in the region of 0 to 10, for example 1 to 5, % by weight of further monomers capable of vinylic copolymerization, such as, for example, hydroxyethyl methacrylate or hydroxy-ethyl acrylate, to be present. It is preferred that no further monomers capable of vinylic copolymerization are present.

C1- to C4-alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group is, for example, acrylic acid, with preference for methacrylic acid.

Suitable anionic (meth)acrylate copolymers are those polymerized from of 40 to 60% by weight methacrylic acid and 60 to 40% by weight methyl methacrylate or 60 to 40% by weight ethyl acrylate (EUDRAGIT® L or EUDRAGIT® L 100 55 types).

EUDRAGIT® L is a copolymer polymerized from 50% by weight methyl methacrylate and 50% by weight methacrylic acid. The pH of the start of the specific active ingredient release in intestinal juice or simulated intestinal fluid can be stated to be at about pH value 6.0. It has a T_{gm} of >125° C.

EUDRAGIT® L 100-55 is a copolymer polymerized from 50% by weight ethyl acrylate and 50% by weight methacrylic acid. EUDRAGIT® L 30 D-55 is a dispersion comprising 30% by weight EUDRAGIT® L 100-55.

Likewise, suitable are anionic (meth)acrylate copolymers polymerized from 20 to 40% by weight methacrylic acid and 80 to 60% by weight methyl methacrylate (EUDRAGIT® S type). The pH value of the start of the specific active ingredient release in intestinal juice or simulated intestinal fluid can be stated to be at about pH value 7.0.

Suitable (meth)acrylate copolymers are polymerized from 10 to 30% by weight methyl methacrylate, 50 to 70% by weight methyl acrylate and 5 to 15% by weight methacrylic acid (EUDRAGIT® FS type).

EUDRAGIT® FS is a copolymer polymerized from 25% by weight methyl methacrylate, 65% by weight methyl acrylate and 10% by weight methacrylic acid. EUDRAGIT® FS 30 D is a dispersion comprising 30% by weight EUDRAGIT® FS.

Suitable is a copolymer composed of
20 to 34% by weight methacrylic acid and/or acrylic acid,
20 to 69% by weight methyl acrylate and
0 to 40% by weight ethyl acrylate and/or where appropriate
0 to 10% by weight further monomers capable of vinylic copolymerization,
   with the proviso that the glass transition temperature of the copolymer according to ISO 11357-2:2013-05, subsection 3.3.3, is not more than 60° C.

Suitable is a copolymer polymerized from
20 to 33% by weight methacrylic acid and/or acrylic acid,
5 to 30% by weight methyl acrylate and
20 to 40% by weight ethyl acrylate and
more than 10 to 30% by weight butyl methacrylate and where appropriate
0 to 10% by weight further monomers capable of vinylic copolymerization,
where the proportions of the monomers add up to 100% by weight,
   with the proviso that the glass transition temperature of the copolymer according to ISO 11357-2:2013-05, subsection 3.3.3 (midpoint temperature Tm,g), is 55 to 70° C.

The copolymer preferably consists of 90, 95 or 99 to 100% by weight of the monomers methacrylic acid, methyl acrylate, ethyl acrylate and butyl methacrylate in the ranges of amounts indicated above. However, it is possible, without this necessarily leading to an impairment of the essential properties, for small amounts in the range from 0 to 10, e.g., 1 to 5% by weight of further monomers capable of vinylic copolymerization additionally to be present, such as, for example, methyl methacrylate, butyl acrylate, hydroxyethyl methacrylate, vinylpyrrolidone, vinyl-malonic acid, styrene, vinyl alcohol, vinyl acetate and/or derivatives thereof.

Further suitable anionic (meth)acrylate copolymers can be so called core/shell polymers as described in WO 2012/171575 A2 or WO 2012/171576 A1. A suitable Core Shell polymer is a copolymer from a two-stage emulsion polymerization process with a core of 75% by weight comprising polymerized units of 30% by weight of ethyl acrylate and 70% by weight of methyl methacrylate and a shell of polymerized units comprising 25% by weight of polymerized from 50% by weight ethyl acrylate and 50% by weight methacrylic acid.

A suitable Core-Shell polymer can be a copolymer from a two-stage emulsion polymerization process with a core with 70 to 80% by weight, comprising polymerized units of 65 to 75% by weight of ethyl acrylate and 25 to 35% by weight of methyl methacrylate, and a shell with 20 to 30% by weight, comprising polymerized units of 45 to 55% by weight ethyl acrylate and 45 to 55% by weight methacrylic acid.

Anionic Celluloses Anionic celluloses can be selected from carboxymethyl ethyl cellulose and its salts, cellulose acetate phthalate (CAP), cellulose acetate succinate (CAS), cellulose acetate trimellitate (CAT), hydroxypropyl methyl cellulose phthalate (HPMCP, HP50, HP55), hydroxypropyl methyl cellulose acetate succinate (HPMCAS-LF, -MF, -HF).

The coating layer can comprise one or more anionic cellulose(s), ethyl cellulose and/or one or more starches comprising at least 35% by weight amylose, preferably with a glass transition temperature T_{gm} of 130° C. or less (determined by Differential Scanning Calorimetry (DSC) according to ISO 11357-2:2013-05), wherein the coating layer is preferably present in an amount of about 1 to 5.8, more preferably 2 to 5 mg/cm2.

The coating layer can comprise in total 10 to 100, 20 to 95, 30 to 90% by weight of one or more anionic cellulose(s), ethyl cellulose and/or one or more starches comprising at least 35% by weight amylose.

The glass transition temperature T_{gm} of hydroxypropyl methyl cellulose phthalate is about 132 to 138° C. (type HP-55 about 133° C., type HP-50 about 137° C.).

The glass transition temperature T_{gm} of hydroxypropyl methyl cellulose acetate succinate (HPMCAS) is about 120° C. (AquaSolve™ L HPMCAS 119° C., AquaSolve™ M HPMCAS 120° C., AquaSolve™ H HPMCAS 122° C.).

### Anionic Vinyl Copolymers

Anionic vinyl copolymers can be selected from unsaturated carboxylic acids other than acrylic acid or methacrylic acid as exemplified by polyvinylacetatephthalate or a copolymer of vinylacetate and crotonic acid (preferably at a ratio of 9:1).

### Cationic Polymers

A suitable cationic (meth)acrylate copolymer comprised in the coating layer can be polymerized from monomers comprising C1- to C4-alkyl esters of acrylic or of methacrylic acid and an alkyl ester of acrylic or of methacrylic acid with a tertiary or a quaternary ammonium group in the alkyl group. The cationic, water-soluble (meth)acrylate copolymer can be polymerized partly or fully of alkyl from acrylates and/or alkyl methacrylates having a tertiary amino group in the alkyl radical. A coating comprising these kinds of polymers may have the advantage of providing moisture protection to the hard shell capsule. Moisture protection shall be understood a reduced uptake of moisture or water during storage of the readily filled and final-locked capsules.

A suitable cationic (meth)acrylate copolymer can be polymerized from 30 to 80% by weight of C1- to C4-alkyl esters of acrylic or of methacrylic acid, and 70 to 20% by weight of alkyl(meth)acrylate monomers having a tertiary amino group in the alkyl radical.

The preferred cationic (meth)acrylate copolymer can be polymerized from 20-30% by weight of methyl methacrylate, 20-30% by weight of butyl methacrylate and 60-40% by weight of dimethylaminoethyl methacrylate (EUDRAGIT® E type polymer).

A specifically suitable commercial (meth)acrylate copolymer with tertiary amino groups is polymerized from 25% by weight of methyl methacrylate, 25% by weight of butyl methacrylate and 50% by weight of dimethylaminoethyl methacrylate (EUDRAGIT® E 100 or EUDRAGIT® E PO (powder form)). EUDRAGIT® E 100 and EUDRAGIT® E PO are water-soluble below approx. pH value 5.0 and are thus also gastric juice-soluble.

A suitable (meth)acrylate copolymer can be composed of 85 to 98% by weight of free-radical polymerized C1 to C4 alkyl esters of acrylic or methacrylic acid and 15 to 2% by weight of (meth)acrylate monomers with a quaternary amino group in the alkyl radical.

Preferred C1 to C4 alkyl esters of acrylic or methacrylic acid are methyl acrylate, ethyl acrylate, butyl acrylate, butyl methacrylate and methyl methacrylate.

Further suitable cationic (meth)acrylate polymers may contain polymerized monomer units of 2-trimethylammonium-ethyl methacrylate chloride or trimethylammonium-propyl methacrylate chloride.

An appropriate copolymer can be polymerized from 50 to 70% by weight of methyl methacrylate, 20 to 40% by weight of ethyl acrylate and 7 to 2% by weight of 2-trimethylammoniumethyl methacrylate chloride.

A specifically suitable copolymer is polymerized from 65% by weight of methyl methacrylate, 30% by weight of ethyl acrylate and 5% by weight of 2-trimethylammoniumethyl methacrylate chloride (EUDRAGIT® RS).

A further suitable (meth)acrylate copolymer can be polymerized from 85 to less than 93% by weight of C1 to C4 alkyl esters of acrylic or methacrylic acid and more than 7 to 15% by weight of (meth)acrylate monomers with a quaternary amino group in the alkyl radical. Such (meth)acrylate monomers are commercially available and have long been used for release-slowing coatings.

A specifically suitable copolymer is polymerized from 60% by weight of methyl methacrylate, 30% by weight of ethyl acrylate and 10% by weight of 2-trimethylammoniumethyl methacrylate chloride (EUDRAGIT® RL).

### Neutral Polymers

Neutral polymers are defined as polymers which are polymerized from neutral monomers and less than 5, preferably less than 2% by weight or most preferred no monomers with ionic groups.

Suitable neutral polymers for the coating of the hard shell capsule are methacrylate copolymers, preferably copolymers of ethyl acrylate and methyl methacrylate like EUDRAGIT® NE or EUDRAGIT® NM, neutral celluloses, such as methyl-, ethyl- or propyl ethers of cellulose, for instance hydroxypropyl cellulose, polyvinyl pyrrolidone, polyvinyl acetate or polyvinyl alcohol.

Neutral methacrylate copolymers are often useful in mixture with anionic (meth)acrylate copolymers.

Neutral methacrylate copolymers are polymerized from at least to an extent of more than 95% by weight, in particular to an extent of at least 98% by weight, preferably to an extent of at least 99% by weight, in particular to an extent of at least 99% by weight, more preferably to an extent of 100% by weight, of (meth)acrylate monomers with neutral radicals, especially C1- to C4-alkyl radicals.

Suitable (meth)acrylate monomers with neutral radicals are, for example, methyl methacrylate, ethyl methacrylate, butyl methacrylate, methyl acrylate, ethyl acrylate, butyl acrylate. Preference is given to methyl methacrylate, ethyl acrylate and methyl acrylate.

Methacrylate monomers with anionic radicals, for example acrylic acid and/or methacrylic acid, can be present in small amounts of less than 5% by weight, preferably not more than 2% by weight, more preferably not more than 1 or 0.05 to 1% by weight.

Suitable examples are neutral or virtually neutral (meth)acrylate copolymers polymerized from 20 to 40% by weight of ethyl acrylate, 60 to 80% by weight of methyl methacrylate and 0 to less than 5% by weight, preferably 0 to 2 or 0.05 to 1% by weight of methacrylic acid or acrylic acid.

Suitable examples are neutral or virtually neutral (meth)acrylate copolymers polymerized from 20 to 40% methyl methacrylate by weight of, 60 to 80% by weight of ethyl acrylate and 0 to less than 5% by weight, preferably 0 to 2 or 0.05 to 1% by weight of methacrylic acid or acrylic acid. (EUDRAGIT® NE or EUDRAGIT® NM type).

EUDRAGIT® NE and EUDRAGIT® NM are copolymers comprising free-radically polymerized units of 28 to 32% by weight of methyl methacrylate and 68 to 72% by weight of ethyl acrylate.

Preference is given to neutral or essentially neutral methyl acrylate copolymers which, according to WO 01/68767 A1, have been prepared as dispersions using 1-10% by weight of a non-ionic emulsifier having an HLB value of 15.2 to 17.3. The latter offer the advantage that there is no phase separation with formation of crystal structures by the emulsifier (EUDRAGIT® NM type).

According to EP 1 571 164 A2, corresponding, virtually neutral (meth)acrylate copolymers with small proportions of 0.05 to 1% by weight of monoolefinically unsaturated C3-C8-carboxylic acids can, however, also be prepared by emulsion polymerization in the presence of comparatively small amounts of anionic emulsifiers, for example 0.001 to 1% by weight.

### Natural Polymers

Especially for nutraceutical dosage forms so called “natural polymer” coatings are preferred by many customers. Natural polymers are based on a source from nature, plants, microorganisms or animals, but sometimes further chemically processed. Natural polymers for coatings can be selected from polymers such as starch, alginates or salts of alginates, preferably sodium alginate, pectin, shellac, zein, carboxymethyl-zein, modified starch, for instance EUDRAGUARD® Natural, marine sponge collagen, chitosan, gellan gum. Suitable polymer mixtures may comprise: Ethyl cellulose and pectin, modified starch (EUDRAGUARD® Natural) and alginate and/or pectin, shellac and alginate and/or pectin, shellac and inulin, whey protein and gums (such as guar gum or tragacanth gum), zein and polyethylene glycol, sodium alginate and chitosan.

Ethyl cellulose is a derivative of cellulose in which some of the hydroxyl groups of the repeating glucose units are converted into ethyl ether groups. Ethyl cellulose can be used as a delayed release coating material for the capsules as disclosed. The glass transition temperature T_{gm} of ethyl cellulose can be in the range of about 128 to 130° C. (Hui Ling Lai et al. Int.J.Pharmaceuticals 386 (2010) 178-184).

In the Following Further Components, which can be Present in the Enteric or Top Coat are Described

Unless explicitly state otherwise, the components are in general suitable to be used in both coating layers. The amount of the respective component is indicated in view of the total weight of the at least one polymer, contained in the respective coating layer, unless explicitly stated otherwise.

### Glidants

Glidants usually have lipophilic properties. They prevent agglomeration of cores during film formation of the film forming polymers.

The at least one glidant is preferably selected from silica, for example commercially available under the tradenames RXCIPIENTS® GL100 or RXCIPIENTS® GL200, ground silica, fumed silica, kaolin calcium silicate, magnesium silicate, colloidal silicone dioxide, talc, stearate salts like calcium stearate, magnesium stearate, zinc stearate, sodium stearyl fumarate, starch, stearic acid, preferably talc, magnesium stearate, colloidal silicon dioxide und glycerol monostearate or mixtures thereof, more preferred glycerol monostearate and talc or mixtures thereof.

Standard proportions for use of glidants in the coating layer range between 0.5 and 100% by weight, preferably 3 to 75% by weight, more preferably 5 to 50% by weight, most preferably 5 to 30% by weight, relative to the total weight of the at least one polymer.

### Emulsifiers

In general, all known emulsifiers are suitable. Preferred are non-ionic emulsifier, in particular emulsifier having an HLB>10 or HLB>12. The HBL Value can be determined according to Griffin, William C. (1954), “Calculation of HLB Values of Non-Ionic Surfactants” (PDF), Journal of the Society of Cosmetic Chemists, 5 (4): 249-56.

The at least one emulsifier is preferably selected from polyglycosides, alcohols, sugar and sugar derivatives, polyethers, amines, polyethylene derivatives, alkyl sulfates (e.g., sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and mixtures thereof.

The at least one emulsifier is preferably selected from alkyl polyglycosides, decyl glucoside, decyl polyglucose, lauryl glucoside, octyl glucoside, N-octyl beta-D-thioglucopyranoside, cetostearyl alcohol, cetyl alcohol, stearyl alcohol, polyoxyethylene cetostearyl alcohol, cetylstearyl alcohol, oleyl alcohol, polyglyceryl-8-dioleate, glyceryl stearate citrate, polyglyceryl-3 caprate, polyglyceryl-3 diisostearate, glyceryl isostearate, polyglyceryl-4-isostearate, glyceryl monolinoleate, dicaprylyl carbonate, alcohol polyglycol ether, polyethylenglycolether of cetearylalcohols (n=20), polyethylene glycol-6 stearate, glycol stearate, polyethylene glycol-32 stearate, polyethylene glycol-20 stearate, fatty alcohol polyglycol ether, polyethylene glycol-4 laurate, polyethylene glycol isocetyl ether (n=20), polyethyleneglycol-32 (Mw 1500 g/mol) mono- and diesters of lauric acid (C12), nonaethylene glycol, polyethylene glycol nonylphenyl ether, octaethylene glycol monododecyl ether, pentaethylene glycol monododecyl ether, polyethylene glycol macrocetyl ether, polyethylene glycol esters of palmitic (C16) or stearic (C18) or caprylic acids, polyoxyethylene fatty ether derived from stearyl alcohols like BRIJ S2, polyoxyethylene oxypropylene stearate, macrogol stearyl ether (20), diethylaminoethyl stearate, polyethylene glycol stearate, sucrose distearate, sucrose tristearate, sorbitan monostearate, sorbitan tristearate, mannide monooleate, octaglycerol monooleate, sorbitan dioleate, polyricinoleate, polysorbate like polysorbate 20 and Polyoxyethylene (20) sorbitan monooleate (polysorbate 80), sorbitan, sorbitan monolaurate, sucrose cocoate, glycereth-2 cocoate, ethylhexyl cocoate, polypropylene glycol-3 benzyl ether myristate, sodium myristate, gold sodium thiomalate, polyethylene glycol 8 laurate, polyethylene-4 dilaurate, from α-Hexadecyl-ω-hydroxypoly(oxyethylene), cocamide diethanolamine, N-(2-hydroxyethyl)dodecanamide, octylphenoxypolyethoxyethanol, maltoside, 2,3-Dihydroxypropyl dodecanoate, 3-[(3R,6R,9R,12R,15S,22S,25S,30aS)-6,9,15,22-Tetrakis(2-amino-2-oxoethyl)-3-(4-hydroxybenzyl)-12-(hydroxymethyl)-18-(11-methyltridecyl)-1,4,7,10,13,16,20,23,26-nonaoxotriacontahydropyrrolo[1,2-g][1,4,7,10,13,16,19,22,25]nonaazacyclooctacosin-25-yl]propenamide, 2-{2-[2-(2-{2-[2-(2-{2-[2-(4-nonylphenoxy)ethoxy]ethoxy}ethoxy)ethoxy]ethoxy}ethoxy)ethoxy]ethoxy}ethanol, oxypolyethoxydodecane, poloxamers like poloxamer 188 (Pluronic F-88) and poloxamer 407, propylene glycol monocaprylate, type I (Capryol PGMC), polyethoxylated tallow amine, polyglycerol, polyoxyl 40 hydrogenated castor oil, surfactin, 2-[4-(2,4,4-trimethylpentan-2-yl)phenoxy]ethanol, carbomer, sodium carbomer, carboxymethylcellulose calcium, carrageenan, cholesterol, deoxycholic acid, phospholipids like egg phospholipids, gellan gum, lanolin, capric acid, waxes like Polawax NF, Polawax A31 or Ceral PW, ester gum, dea-cetyl phosphate, soya lecithin, sphingomyelins, sodium phosphate, sodium lauroyl lactylate, lanolin, Oxirane methyl-polymer with oxirane monobutyl ether, 1,2-dierucoylphosphatidycholine, dimethicone end-blocked with an average of 14 moles of propylene oxide, laurylmethicone copolyol, lauroglycol 90, white mineral oil like Amphocerine KS, dispersion of acrylamide/sodium acryloykdimethyl taurate copolymer in isohexadecane, and sodium polyacrylate or mixtures thereof. Preferred are macrogol stearyl ether (20) and polysorbate 80.

In one embodiment less than 3 wt.-%, preferably 1.5 wt.-% of at least one emulsifier based on the total weight of the at least one polymer is present or essentially no or no emulsifier is present.

### Enteric or Top Coating Layer

The enteric or top coating layer may comprise 10% or more, 20% or more, 30% or more, 40% or more, 50% or more, 60% or more, 70% or more, 80% or more, 90% or more by weight or 95% or more by weight of the polymers. The coating layer may comprise 10 to 100, 10 to 90, 12 to 80, 15 to 80, 18 to 80, 20 to 80 or 40 to 80% by weight of the polymers.

The top coating layer is located on top of the enteric coating layer, comprising the at least one polymer as described above. A top coat is also preferably water-soluble or essentially water-soluble. A top coat may have the function of colouring the pharmaceutical or nutraceutical form or protecting from environmental influences for instance from moisture during storage.

### Intermediate Coating Layer

The intermediate coating layer is located upon or above the capsule and comprises at least one polymer and at least one alkaline agent.

The intermediate coating layer can comprise 5 to 90% by weight, preferably 10 to 70% by weight, more preferably 30 to 50% by weight of the at least one polymer based on the total weight of the intermediate coating layer. The at least one polymer is preferably selected selected from celluloses, like hydroxyethyl cellulose (HEC), hydroxypropyl cellulose (HPC), hydroxypropyl methyl cellulose (HPMC), hydroxyethyl methyl cellulose (HEMC), ethyl cellulose (EC), methyl cellulose (MC), cellulose esters, cellulose glycolates, polyethylene glycols, polyethylene oxides, polyvinyl pyrrolidone, polyvinyl acetate, polyvinyl alcohol, or a mixture thereof, preferably is hydroxypropyl methyl cellulose.

The intermediate coating layer can comprise 10 to 75, preferably 10 to 50, % by weight of the alkaline agent, based on the total weight of the coating layer.

The alkaline agent can be an alkali or an earth alkali metal salt. The alkaline agent can be selected from calcium oxide, calcium carbonate, magnesium carbonate, magnesium oxide, sodium carbonate, sodium bicarbonate and sodium hydroxide or any mixtures thereof. Preferred alkaline agents are magnesium oxide and/or magnesium carbonate.

The intermediate coating layer com comprise at least one glidant, preferably in an amount of 2 to 50, more preferably 5 to 25% by weight, based on the total weight of the intermediate coating layer. Suitable glidants are the same as described for the enteric coating layer herein. The at least one glidant is preferably glycerol.

The intermediate layer can comprise 1 to 95, preferably 10 to 50% by weight of at least one additive based on the total weight of the intermediate layer, such as a polymeric binder, a plasticizer or an anti-tacking agent or a combination thereof. Suitable additives are the same as described for the enteric coating layer.

Preferably the intermediate layer is directly located upon the capsule.

### Amount and Thickness of the Enteric and Intermediate Coating Layer

In order to ensure influx prevention and processability of the final capsule in an industrial filling machine, it has been found that the total coating amount is required to be 2.0 to 10 mg/cm.

For a hard shell capsule of size #0, the amount of the coating layer should not be too high. If the amount of coating layer applied is too high this may result in difficulties to process the polymer-coated pre-locked hard shell capsules subsequently in a capsule-filling machine. If the amount of coating layer is less than 5 mg/cm², for instance 2 to 4 mg/cm² usually no problem with standard capsule-filling machines without modification will occur. In the range from 4 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 3 to about 8 mg/cm².

For a hard shell capsule of size #1, the amount of the coating layer should not be too high. If the amount of coating layer applied is too high this may result in difficulties to process the polymer-coated pre-locked hard shell capsules subsequently in a capsule-filling machine. If the amount of coating layer is less than 4 mg/cm², for instance 2 to 3.5 mg/cm² usually no problem with standard capsule-filling machines without modification will occur. In the range from 3.5 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 3 to about 8 mg/cm².

For a hard shell capsule of size #3, the amount of the coating layer should not be too high. If the amount of coating layer applied is too high this may result in difficulties to process the polymer-coated pre-locked hard shell capsules subsequently in a capsule-filling machine. In the range from 2 and up to about 6 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 3 to about 6 mg/cm².

If the amount of coating layer applied is too high there will be also an assembly of too much coating layer at the rim of the cap where the gap between body and cap is in the pre-locked state. This may result after drying in fissures of the coating layer when the coated pre-locked hard shell capsule is opened manually or in a machine. The fissures may result in a later leakage of the capsule. Finally, a too thick coating may result in difficulties or make it impossible to close the opened coated hard shell capsule to the final-locked state since the coating layer is thicker than the gap in the overlapping area between the body and the cap.

As a rough rule the coating layer on the hard shell capsule can be applied in an amount (=a total weight gain) of 0.7 to 20, 1.0-18, 2 to 10, 4 to 8, 1.0 to 8, 1.5 to 5.5, 1.5 to 4 mg/cm².

As a rough rule the coating layer on the hard shell capsule may have an average thickness of about 5 to 100, 10 to 50, 15 to 75 μm.

As a rough rule the coating layer on the hard shell capsule can be applied in an amount of 5 to 50, preferably 8-40% dry weight in relation to the weight of the pre-locked capsule.

With this guidance a skilled person will be able to adjust the amounts of the coating layer in a range between too low and too high.

### Biologically Active Ingredient

The biologically active ingredient is preferably a pharmaceutical active ingredient and/or a nutraceutical active ingredient and/or a cosmetically active ingredient. Even though it is possible that certain biologically active ingredients are contained in the respective coating layers, it is preferred that the biologically active ingredient is contained in the fill. In particular, if the biologically active ingredient is comprised in a liposome, lipid nanopartide or nucleic acid, the biologically active ingredient is only contained in the fill.

### Pharmaceutical or Nutraceutical Active Ingredients

The invention is particularly useful for delayed release formulated pharmaceutical or nutraceutical dosage forms with a fill of pharmaceutical or nutraceutical active ingredients.

Suitable therapeutic and chemical classes of pharmaceutical active ingredients which members can be used as fill for the described polymer-coated hard shell capsules are for instance: analgesics, antibiotics or anti-infectives, antibodies, antiepileptics, antigens from plants, antirheumatics, benzimidazole derivatives, beta-blocker, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptide, hormones, proteins, therapeutic bacteria, peptides, proteins (metal)salt i.e. aspartates, chlorides, urology drugs, lipid nanoparticles, liposomes, polymeric nanoparticles, vaccines. In a preferred embodiment at least one liposome or lipid nanoparticle each comprising at least one polynucleotide is contained.

In a preferred embodiment the pharmaceutically active ingredient is a lipid nanoparticle or liposome each comprising a polynucleotide or a nucleic acid, more preferably a nucleic acid agent can be DNA, RNA, or combinations thereof. In some embodiments, a nucleic acid agent can be an oligonucleotide and/or polynucleotide. In some embodiments, a nucleic acid agent may be an oligonucleotide and/or modified oligonucleotide (including, but not limited to, modifications through phosphorylation); an antisense oligonucleotide and/or modified antisense oligonucleotide (including, but not limited to, modifications through phosphorylation). In some embodiments, a nucleic acid agent can comprise cDNA and/or genomic DNA. In some embodiments, a nucleic acid agent can comprise non-human DNA and/or RNA (e.g., viral, bacterial, or fungal nucleic acid sequences). In some embodiments, a nucleic acid agent can be a plasmid, cosmid, gene fragment, artificial and/or natural chromosome (e.g., a yeast artificial chromosome), and/or a part thereof. In some embodiments, a nucleic acid agent can be a functional RNA (e.g., mRNA, a tRNA, an rRNA and/or a ribozyme). In some embodiments, a nucleic acid agent can be an RNAi-inducing agent, small interfering RNA (siRNA), short hairpin RNA (shRNA), and/or microRNA (miRNA). In some embodiments, a nucleic acid agent can be a peptide nucleic acid (PNA). In some embodiments, a nucleic acid agent can be a polynucleotide comprising synthetic analogues of nucleic acids, which may be modified or unmodified. In some embodiments, a nucleic acid agent can comprise various structural forms of DNA including single-stranded DNA, double-stranded DNA, supercoiled DNA and/or triple-helical DNA; Z-DNA; and/or combinations thereof. Further suitable nucleic acids are for example disclosed in WO 2012103035 A1, which are incorporated by reference.

Further examples of drugs that can be used as fill for the described polymer-coated hard shell capsules are for instance acamprosat, aescin, amylase, acetyisalicylic acid, adrenalin, 5-amino salicylic acid, aureomycin, bacitracin, balsalazine, beta carotene, bicalutamid, bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexiansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interieukin, Intron A, ketoprofen, lansoprazole, leuprolidacetat lipase, lipoic acid, lithium, kinin, memantine, mesalazine, methenamine, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, acetaminophen, codeine, griseofulvin, isosorbide-5-mononitrate, lumiracoxib, metoprolol, minoxidil, nefazodone, phenytoin, remogliflozin etabonate, rivastigmine, theophylline, colchicine, darunavir, nifedipine, valacyclovir, mizoribine, ribavirin, antipyrine, glipizine, phenytoin, selegiline, theophylline, cilostazol, cimetidine, darunavir, digoxin, famotidine, fexofenadine, forskolin, indinavir, nevirapine, quinidine, ranitidine, tacrolimus, talinolol, verapamil, benazepril, cefadroxil, cephalexin, atenolol, ciprofloxacin, Fluvastatin, metformin, levodopa, gabapentin, pseudoephedrine, ropivacaine, sotalol, azacytidine and decitabine or mixtures or combinations thereof, preferably acetaminophen, codeine, griseofulvin, isosorbide-5-mononitrate, lumiracoxib, metoprolol, minoxidil, nefazodone, phenytoin, remogliflozin etabonate, rivastigmine, theophylline, colchicine, darunavir, nifedipine, valacyclovir, mizoribine, ribavirin, antipyrine, glipizine, phenytoin, selegiline, theophylline, cilostazol, cimetidine, darunavir, digoxin, famotidine, fexofenadine, forskolin, indinavir, nevirapine, quinidine, ranitidine, tacrolimus, talinolol, verapamil, benazepril, cefadroxil, cephalexin, atenolol, ciprofloxacin, Fluvastatin, metformin, levodopa, gabapentin, pseudoephedrine, ropivacaine, sotalol, azacytidine and decitabine or mixtures or combinations thereof.

It is evident to a skilled person that there is a broad overlap between the terms pharmaceutical and nutraceutical active ingredients, excipients and compositions respectively a pharmaceutical or a nutraceutical dosage form. Many substances listed as nutraceuticals may also be used as pharmaceutical active ingredients. Depending on the specific application and local authority legislation and classification, the same substance can be listed as a pharmaceutical or a nutraceutical active ingredient respectively a pharmaceutical or a nutraceutical composition or even both.

Nutraceuticals are well known to the skilled person. Nutraceuticals are often defined as extracts of foods claimed to have medical effects on human health. Thus, nutraceutical active ingredients may display pharmaceutical activities as well: Examples for nutraceutical active ingredients can be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus, it is clear that many substances listed as nutraceuticals may also be used as pharmaceutical active ingredients.

Typical nutraceuticals or nutraceutical active ingredients that can be used as fill for the described polymer-coated hard shell capsules may also include probiotics and prebiotics. Probiotics are living microorganisms believed to support human or animal health when consumed. Prebiotics are nutraceuticals or nutraceutical active ingredients that induce or promote the growth or activity of beneficial microorganisms in the human or animal intestine.

Examples for nutraceuticals are resveratrol from grape products, omega-3-fatty acids or pro-anthocyanines from blueberries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals or nutriceuticals are used as synonyms for nutraceuticals.

Preferred biologically active ingredients are azacytidine, decitabine, metoprolol, mesalamine and omeprazole.

### Additives

Additives according to the present invention are preferably excipients, which are well known to a skilled person and often formulated along with the biologically active ingredient contained in the coated hard shell capsule and/or with a polymer coating layer of the hard shell capsule as disclosed and claimed herein. All excipients used must be toxicologically safe and be used in pharmaceuticals or nutraceuticals without risk for patients or consumers.

The dosage form may comprise excipients, preferably pharmaceutical or nutraceutical acceptable excipients, selected from the group of antioxidants, brighteners, binding agents, flavouring agents, flow aids, fragrances, penetration-promoting agents, pigments, pore-forming agents or stabilizers or combinations thereof. The pharmaceutically or nutraceutically acceptable excipients can be comprised in the core and/or in the coating layer comprising the polymer as disclosed. A pharmaceutical or nutraceutical acceptable excipient is an excipient, which is allowed to be used for the application in the pharmaceutical or nutraceutical field.

The intermediate, enteric, or top coating layer may comprise up to 90, up to 80, up to 70, up to 50, up to 60, up to 50, up to 40, up to 30, up to 20, up to 10, up to 5% up to 3%, up to 1% by weight or not any (0%) additives at all, preferably pharmaceutically or nutraceutically acceptable excipients, based on the total weight of the at least one polymer.

### Plasticizers

The polymer coating of the hard shell capsule may comprises one or more plasticizers. Plasticizers achieve through physical interaction with a polymer a reduction in the glass transition temperature and promote film formation, depending on the added amount. Suitable substances usually have a molecular weight of between 90 and 20,000 g/mol and comprise one or more hydrophilic groups in the molecule, e.g. hydroxyl, ester or amino groups.

Examples of suitable plasticizers are alkyl citrates, alkyl phthalates, alkyl sebacates, diethyl sebacate, dibutyl sebacate, polyethylene glycols, and polypropylene glycols. Preferred plasticizers are triethyl citrate (TEC), acetyl triethyl citrate (ATEC), diethyl sebacate, dibutyl sebacate (DBS), polyethylene glycols, and polypropylene glycols or mixtures thereof.

Addition of the plasticizers to the formulation can be carried out in a known manner, directly, in aqueous solution or after thermal pre-treatment of the mixture. It is also possible to employ mixtures of plasticizers. The polymer coating of the hard shell capsule may comprise one or more plasticizers, preferably up to 60, up to 30, up to 25, up to 20, up to 15, up to 10, up to 5, less than 5% by weight, calculated on the at least one polymer, of a plasticizer or any (0%) plasticizer at all can be comprised.

### Fillers

Standard fillers are usually added to the inventive formulation during processing to coating and binding agents. The quantities introduced and the use of standard fillers in pharmaceutical coatings or over layers is familiar to those skilled in the art. Examples of standard fillers are release agents, pigments, stabilizers, antioxidants, pore-forming agents, penetration-promoting agents, brighteners, fragrances or flavoring agents. They are used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary, as an additional control parameter.

### Pigments

Only rarely a pigment is added in soluble form. As a rule, pigments, such as aluminum oxide or iron oxide pigments are used in dispersed form. Titanium dioxide is used as a whitening pigment. Standard proportions for use of pigments are between 10-200, 20-200% by weight relative to the total weight of the at least one polymer in the coating layer. Proportions up to 200% by weight based on the total weight of the at least one polymer can be easily processed.

In a particularly advantageous embodiment, the pigment is used in the top coat. Application takes place in the form of powder or by spraying from aqueous suspension with 5 to 35% (w/w) solid content. The necessary concentration is lower than for incorporation into the polymer layer and amounts to 0.1 to 2% by weight relative to the weight of the pharmaceutical form.

### Process for Preparing a Coated Hard Shell Capsule

Described is a process for preparing a polymer-coated hard shell capsule, wherein the hard shell capsule comprises a body and a cap, wherein in the dosed state the cap overlaps the body either in a pre-locked state or in a final-locked state, wherein the hard shell capsule is provided in the pre-locked state and coated, preferably spray-coated, with the first coating solution, suspension or dispersion according to the present invention to create an intermediate coating layer, then optionally dried and coated, preferably spray-coated, with a coating solution, suspension or dispersion according to the present invention to create an enteric coating layer, which covers the outer surface of the hard shell capsule in the pre-locked state. Furthermore, a top coating layer can subsequently, after an optional drying step be applied.

In a further process step the pre-locked hard shell capsule can be provided with a fill comprising a pharmaceutical or a nutraceutical biologically active ingredient and is closed to the final-locked state.

In such a further process step the polymer-coated hard shell capsule in the pre-locked state can be opened, filled with a fill comprising at least one biologically active ingredient, and is closed in the final-locked state. This further process step is preferably performed in that the coated hard shell capsule in the pre-locked state is provided to a capsule-filling machine, which performs the opening, filling with a fill comprising at least one biologically active ingredient and closing of the polymer-coated hard shell capsule to the final-locked state.

This further process step results in a final-locked polymer-coated hard shell capsule, which is a container for at least one biologically active ingredient. The final-locked polymer-coated hard shell capsule, which as a container for at least one biologically active ingredient is preferably a pharmaceutical or nutraceutical dosage form.

The pharmaceutical or nutraceutical dosage form preferably comprises a polymer-coated hard shell capsule in the final-locked state containing a fill comprising at least one biologically active ingredient, wherein the polymer-coated hard shell capsule comprises coating layers according to the invention, where the coating layers cover the outer surface area of the capsule in the pre-locked state but not the overlapping area where the cap covers the body in the pre-locked state.

A coating suspension can contain an organic solvent, for instance acetone, iso-propanol or ethanol. The concentration of dry weight material in the organic solvent can be about from 5 to 50% by weight of polymer. A suitable spraying concentration can be about 5 to 25% by dry weight.

A coating suspension can be the dispersion of polymers in an aqueous medium, for instance water or a mixture of 80% by weight or more of water and 20% or less by weight of water-soluble solvents, such as acetone or isopropanol. A suitable concentration of dry weight material in the aqueous medium can be from about 5 to 50% by weigh. A suitable spraying concentration can be about 5 to 25% by dry weight.

The spray coating is preferably performed by spraying the coating solution or dispersion onto the pre-locked capsules in a drum coater or in a fluidized bed coating equipment.

### Process for Preparing a Fill for the Dosage Form

Suitable processes for preparing the fill for the pharmaceutical or nutraceutical dosage form are well known to a skilled person. A suitable process for preparing the fill for the pharmaceutical or nutraceutical dosage form as disclosed herein can be by forming a core comprising the biologically active ingredient in the form of mini-tablets by direct compression, compression of dry, wet or sintered granules, by extrusion and subsequent rounding off, by wet or dry granulation, by direct pelleting or by binding powders onto active ingredient-free beads or neutral cores or active ingredient-containing particles or pellets and optionally by applying coating layers in the form of aqueous dispersions or organic solutions in spray processes or by fluidized bed spray granulation.

### Capsule Filling Machine

The polymer-coated hard-shell capsule is provided in the pre-locked state to a capsule-filling machine, which performs the steps of separating the body and the cap, filling the body with the fill and rejoining the body and the cap in the final-locked state.

The capsule filling machine used can be a capsule filling machine, preferably a fully automated capsule filling machine, that is capable to produce filled and closed capsules at a speed with an output of 1,000 or more filled and finally closed capsules per hour. Capsule filling machines, preferably fully automated capsule filling machines, are well known in the art and commercially available from several companies.

The capsule filling machine used can be preferably operated at a speed with an output of 1,000 or more, preferably 10,000 or more, 100,000 or more, 10,000 up to 500,000, filled and finally closed capsules per hour.

### Capsule Filling Machine General Operations

Before the capsule filling process, the capsule filling machine is provided with a sufficient number or amount of pre-coated hard-shell capsules in the pre-locked state. The capsule filling machine is also provided with sufficient amounts of fill to be filled in during operation.

The hard-shell capsules in the pre-locked state may fall by gravity into feeding tubes or chutes. The capsules can be uniformly aligned by mechanically gauging the diameter differences between the cap and the body. The hard-shell capsules are then usually fed, in proper orientation, into a two-section housing or brushing.

The diameter of the upper bushing or housing is usually larger than the diameter of the capsule body bushing; thus, the capsule cap can be retained within an upper bushing while the body is pulled into a lower bushing by vacuum. Once the capsule is opened/the body and the cap are separated, the upper and lower housing or bushing are separated to position the capsule body for filling.

The open capsule body is then filled with the fill. Various types of filling mechanisms can be applied, with respect to the different fillings such as granules, powders, pellets or mini-tablets. Capsule filling machines in general employ a variety of mechanisms to handle the various dosage ingredients as well as various numbers of filling stations. The dosing systems are usually based on volumetric or amounts of fills governed by the capsule size and capacity of the capsule body. The empty capsule manufacturers usually provide reference tables that indicate the volume capacity of their capsule body and the maximum fill weight for different capsule sizes based on the density of the fill material. After filling, the body and the cap are rejoined by the machine in the final-locked state or position.

### Use/Method of Use/Method Steps

The process for preparing a polymer-coated hard shell capsule suitable as described herein can be understood as a method of use of a hard shell capsule comprising a body and a cap, wherein in the closed state the cap overlaps the body either in a pre-locked state or in a final-locked state, for preparing a polymer-coated hard shell capsule, suitable as container for pharmaceutical or nutraceutical biologically active ingredients, comprising the steps of
a) providing the hard shell capsule is provided in the pre-locked state and
b) spray-coating with a first and second coating solution, suspension or dispersion comprising a polymer or a mixture of polymers to create an intermediate and an enteric coating layer which covers the outer surface of the hard shell capsule in the pre-locked state.

The spray-coating can be preferably applied by using a drum coater equipment or a fluidized bed coating equipment, more preferably by using a drum coater equipment. A suitable product temperature during the spray-coating process can be in the range from about 15 to 40, preferably from about 23 to 30° C. A suitable spray rate can be in the range from about 0.3 to 17.0, preferably 0.5 to 14 [g/min/kg]. After spray-coating a drying step is included.

The polymer-coated hard shell capsule in the pre-locked state can be opened in a step c), filled with a fill comprising a pharmaceutical or a nutraceutical biologically active ingredient in a step d), and is then closed in a step e) to the final-locked state.

Steps c) to e) can be performed manually or preferably supported by a suitable equipment, for instance a capsule-filling machine. Preferably, the coated hard shell capsule in the pre-locked state is provided to a capsule-filling machine, which performs the opening step c), the filling with a fill comprising a pharmaceutical or a nutraceutical biologically active ingredient in step d) and the closing of the capsule to the final-locked state in step e).

The selection of the processes in all their generic or specific features and embodiments as disclosed herein can be combined without restriction with any other generic or specific selections of materials or numerical features and embodiments as disclosed herein, such as polymers, capsule materials, capsule sizes, coating thicknesses, biologically active ingredients and any other embodiments as disclosed.

### Use

The polymer-coated hard shell capsule according to the present invention can be used for providing less than 10% drug release at 0.1N HCl for 120 minutes followed by at least 80% drug release at pH value 5 within additional 150 min.

Drug Release can be Determined in Accordance with Following Conditions.

### A) Dissolution Conditions

### 1) Dissolution Parameters

Apparatus: USP Type I
Dissolution Medium: 2 hours acid stage medium followed by full change to 2 hours buffer stage medium
Sampling points: Acid stage medium; 60 min, 120 min
   : Buffer stage medium; 10 min, 20 min, 30 min, 45 min, 60 min, 120 min
Volume of Medium: 750 mL for acid stage, 1000 mL for buffer stage
Speed: 100 rpm
Temperature: 37° C.±0.5° C.
Withdrawal Volume: 10 ml

### 2) Dissolution Mediums

I. Acid stage medium—0.1 N HCl; buffer stage medium—pH 5.0 buffer, or
II. Acid stage medium—0.1 N HCl; buffer stage medium—pH 4.5 buffer, or
III. Acid stage medium—0.1 N HCl; buffer stage medium—pH 3.0 buffer

### 3) Composition of Buffer Stage Mediums

### I. Buffer Stage Medium—pH 5.0 Buffer

1 g of Potassium dihydrogen phosphate (KH₂PO₄), 2 g of Di-potassium hydrogen phosphate anhydrous (K₂HPO₄) and 8.5 g of sodium chloride (NaCl) was weighed and transferred to 1-liter beaker. To this, 500 mL water was added, salts were dissolved, and volume was made up to 1000 mL with water. The pH was adjusted to 5.0+0.05 using ortho-phosphoric acid.

### II. Buffer Stage Medium—pH 4.5 Buffer

1 g of Potassium dihydrogen phosphate (KH₂PO₄), 2 g of Di-potassium hydrogen phosphate anhydrous (K₂HPO₄) and 8.5 g of sodium chloride (NaCl) was weighed and transferred to 1-liter beaker. To this, 500 mL water was added, salts were dissolved, and volume was made up to 1000 mL with water. The pH was adjusted to 4.5+0.05 using ortho-phosphoric acid.

### III. Buffer Stage Medium—pH 3.0 Buffer

1 g of Potassium dihydrogen phosphate (KH₂PO₄), 2 g of Di-potassium hydrogen phosphate anhydrous (K₂HPO₄) and 8.5 g of sodium chloride (NaC) was weighed and transferred to 1-liter beaker. To this, 500 mL water was added, salts were dissolved, and volume was made up to 1000 mL with water. The pH was adjusted to 3.0+0.05 using ortho-phosphoric acid.

### 4) Dissolution Procedure:

Acid Stage: Accurately weighed six caffeine capsules were transferred in different dissolution jars and then the dissolution test was performed as per parameters given in the method above (Acid Stage). After 1 & 2 hours 10 mL of aliquot was removed and analyzed as acid stage sample solution.

Buffer Stage: The capsules after acid stage were transferred to buffer stage medium. The dissolution test was continued as per parameters given in the method above (Buffer Stage). The aliquots of each interval ware filtered through 0.45 μm nylon membrane syringe filter discarding first few mL of the filtrate and analyzed as buffer stage sample solution using following chromatographic conditions.

### B) Chromatographic Conditions

Column: Agilent Zorbax Eclipse XDB C 18 column, 150×4.6 mm, 5 μm or equivalent
Mobile Phase: Water/ACN (80:20)
Wavelength: 273 nm
Column Temp: 25° C.
Injection Volume: 10 μL
Flow rate: 1.5 mL/minute

### Items

In particular, the present invention refers to:

1. Process for preparing a polymer-coated hard shell capsule comprising at least an intermediate coating layer and an enteric coating layer and optionally a top coating layer, suitable as container for pharmaceutical or nutraceutical biologically active ingredients, wherein the hard shell capsule comprises a body and a cap, wherein in the closed state the cap overlaps the body either in a pre-locked state or in a final-locked state, wherein the hard shell capsule is provided in the pre-locked state and
is coated
with a first coating solution, suspension or dispersion comprising or consisting of
a1) at least one polymer;
b1) at least one alkaline agent;
c1) optionally at least one glidant; and
d1) optionally at least one additive, different from a1) to c1);
to obtain the intermediate coating layer of the hard shell capsule in the pre-locked state; and thereafter
is coated with a second coating solution, suspension or dispersion, which is different from the first coating solution, suspension or dispersion, comprising or consisting of
a2) at least two polymers;
b2) optionally at least one glidant;
c2) optionally at least one emulsifier;
d2) optionally at least one plasticizer;
e2) optionally at least one biologically active ingredient; and
f2) optionally at least one additive, different from a2) to e2);
wherein the first polymer is an anionic polymer having a T_{gm}≥35° C., preferably 35 to 155° C., more preferably 80 to 145° C., most preferably 90 to 125° C.;
wherein the second polymer is a polymer having a T_{gm} of ≤30° C., preferably <15° C.; to obtain the enteric coating layer of the hard shell capsule in the pre-locked state, wherein the total coating amount of the coating layers is 2.0 to 10 mg/cm², preferably 2.2 to 9 mg/cm²; more preferably 2.5 to 8 mg/cm²; and preferably
the coating amount of the intermediate coating layer is at most 150%, at most 100%, preferably at most 80% of the coating amount of the enteric coating layer.

2. Process according to item 1, wherein the base material of the body and the cap is selected from hydroxypropyl methyl cellulose, starch, gelatin, pullulan and a copolymer of a C1- to C4-alkylester of (meth)acrylic acid and (meth)acrylic acid, preferably is hydroxypropyl methyl cellulose.

3. Process according to item 1 or 2, wherein the at least one polymer a1) and/or the at least two polymers a2)
is/are selected from at least one (meth)acrylate copolymer, hydroxypropylmethylcellulose (HPMC), or hydroxypropylcellulose (HPC).

4. Process according to any of the preceding items, wherein the first polymer a2) is
i) a Core-Shell polymer, which is a copolymer obtained by a two stage emulsion polymerization process with a core with 70 to 80% by weight, comprising polymerized units of 65 to 75% by weight of ethyl acrylate and 25 to 35% by weight of methyl methacrylate, and a shell with 20 to 30% by weight, comprising polymerized units of 45 to 55% by weight ethyl acrylate and 45 to 55% by weight methacrylic acid; or
ii) an anionic polymer obtained by polymerizing 25 to 95% by weight C1- to C12-alkyl esters of acrylic acid or of methacrylic acid and 75 to 5% by weight (meth)acrylate monomers with an anionic group; or
iii) a (meth)acrylate copolymer obtained by polymerizing methacrylic acid and ethyl acrylate, methacrylic acid and methyl methacrylate, ethyl acrylate and methyl methacrylate or methacrylic acid, methyl acrylate and methyl methacrylate; or
iv) a (meth)acrylate copolymer obtained by polymerizing 40 to 60% by weight of methacrylic acid and 60 to 40% by weight of ethyl acrylate; or
v) a (meth)acrylate copolymer obtained by polymerizing 5 to 15% by weight methacrylic acid, 60 to 70% by weight of methyl acrylate and 20 to 30% by weight methyl methacrylate; or mixtures thereof.

5. Process according to any of the preceding items, wherein the second polymer a2) is
i) a (meth)acrylate copolymer obtained by polymerizing at least two C1- to C4-alkyl acrylate monomers and 0 to less than 5% by weight of methacrylic acid or acrylic acid; or
ii) a (meth)acrylate copolymer obtained by polymerizing 20 to 40% by weight of ethyl acrylate, 60 to 80% by weight of methyl methacrylate and 0 to less than 5% by weight of methacrylic acid or acrylic acid; or
iii) a (meth)acrylate copolymer obtained by polymerizing 60 to 80% of ethyl acrylate and 40 to 20% by weight of methyl methacrylate.

6. Process according to any of the preceding items, wherein the at least two polymers a2) are
a (meth)acrylate copolymer obtained by copolymerizing 40 to 60% by weight of methacrylic acid and 40 to 60% by weight of ethyl acrylate and
a (meth)acrylate copolymer obtained by polymerizing 60 to 78% of ethyl acrylate and 20 to 38% by weight of methyl methacrylate and optionally up to 2% by weight, preferably 0.1 to 2% by weight of (meth)acrylic acid.

7. Process according to any of the preceding items, wherein the at least one polymer al)
i) is selected from celluloses, like hydroxyethyl cellulose (HEC), hydroxypropyl cellulose (HPC), hydroxypropyl methyl cellulose (HPMC), hydroxyethyl methyl cellulose (HEMC), ethyl cellulose (EC), methyl cellulose (MC), cellulose esters, cellulose glycolates, polyethylene glycols, polyethylene oxides, polyvinyl pyrrolidone, polyvinyl acetate, polyvinyl alcohol, or a mixture thereof, preferably is hydroxypropyl methyl cellulose; and/or
ii) is present in 5 to 90% by weight, preferably 10 to 70% by weight, more preferably 30 to 50% by weight, based on the total weight of the intermediate layer.

8. Process according to any of the preceding items, wherein the at least one alkaline agent in the first coating solution
i) is selected from calcium oxide, calcium carbonate, magnesium carbonate, magnesium oxide, sodium carbonate, sodium bicarbonate and sodium hydroxide or any mixtures thereof, preferably magnesium oxide or magnesium carbonate, more preferred is magnesium oxide; and/or
ii) is present in 10 to 75% by weight, preferably 15 to 50% by weight, based on the total weight of the intermediate layer.

9. Process according to any of the preceding items, wherein in the first coating solution the at least one polymer to the at least one alkaline agent is present in a weight ratio of 1:1 to 1:0.25.

10. Process according to any of the preceding items, wherein at least one glidant is present in the first and/or second coating solution, suspension or dispersion, wherein preferably the at least one glidant
i) is present in an amount of 3 to 75% by weight, based on the total weight of the polymers in the respective coating layer and/or
ii) is selected from silica, ground silica, fumed silica, kaolin calcium silicate, magnesium silicate, colloidal silicone dioxide, glycerol, glycerol monostearate, talc, stearate salts, sodium stearyl fumarate, starch, stearic acid or mixtures thereof, preferably talc, magnesium stearate, colloidal silicon dioxide, glycerol and glycerol monostearate or mixtures thereof, more preferred, glycerol, glycerol monostearate and talc, most preferred is glycerol or glycerol monostearate.

11. Process according to any of the preceding items, wherein at least one emulsifier is present in the second coating solution, suspension or dispersion, wherein preferably the at least one emulsifier
i) is present in an amount of less than 3% by weight, preferably less than 1.5% by weight, based on the total weight of the polymers or is absent; or
ii) is present in an amount of 1.5 to 40% by weight, based on the total weight of the polymers; and/or
iii) is a non-ionic emulsifier, preferably a non-ionic emulsifier having an HLB>10, preferably >12; and/or
iv) is selected from polyglycosides, alcohols, sugar and sugar derivatives, polyethers, amines, polyethylene derivatives, alkyl sulfates (e.g., sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and mixtures thereof.

12. Process according to any of the preceding items, wherein at least one plasticizer is present in the second coating solution, suspension or dispersion, wherein preferably the at least one plasticizer
i) is present in an amount of 2 to 40% by weight, based on the total weight of the polymers and/or
ii) is selected from alkyl citrates, alkyl phthalates, and alkyl sebacates or mixtures thereof, preferably diethyl sebacate, triethyl citrate (TEC), acetyl triethyl citrate (ATEC), diethyl sebacate and dibutyl sebacate (DBS) or mixtures thereof, more preferably is triethyl citrate (TEC).

13. Process according to any of the preceding items, wherein up to 400% by weight, based on the total weight of the at least one polymer, of at least one additive are comprised in the first and/or second coating solution, suspension or dispersion, preferably selected from antioxidants, brighteners, flavouring agents, flow aids, fragrances, penetration-promoting agents, pigments, pore-forming agents or stabilizers, or combinations thereof.

14. Process according to any of the preceding items, wherein the body and the cap are comprising encircling notches or dimples in the area where the cap overlaps the body, that allow the capsule to be closed by a snap-into-place mechanism either in the pre-locked state or in the final-locked state.

15. Process according to any of the preceding items, wherein the body comprises a tapered rim.

16. Process according to any of the preceding items, wherein the polymer-coated hard shell capsule in the pre-locked state is opened, filled with a fill comprising a pharmaceutical or a nutraceutical biologically active ingredient, and is closed to the final-locked state.

17. Process according to any of the preceding items, wherein the polymer-coated hard shell capsule in the pre-locked state is provided to a capsule-filling machine, which performs opening, filling with a fill comprising a pharmaceutical or a nutraceutical biologically active ingredient and closing to the final-locked state.

18. Polymer-coated hard shell capsule, obtained from a process according to any of items 1 to 17.

19. Use of the polymer-coated hard shell capsule according to item 18 for providing less than 10% drug release at 0.1N HCl for 120 minutes followed by at least 80% drug release at pH value 5 within additional 150 min.

TABLE 1
Biologically active ingredients and excipients used in examples

| Sr. | | | | |
|---|---|---|---|---|
| No. | Ingredient | Chemical Name | Pharmacopoeia Grade | Manufacturer/Supplier |
| 1 | Caffeine anhydrous | Caffeine anhydrous | USP | Aarti Industries Limited |
| 2 | VIVAPUR ® 101 | Microcrystalline Cellulose (MCC) | USP/NF, Ph. Eur., JP | JRS PHARMA GmbH + Co. KG |
| 3 | Sotalol HCl | Sotalol Hydrochloride | USP | Panchsheel Organics LTD |
| 4 | PHARM-A-SPHERES (MESH 20-25) | Sugar spheres, 710-850 microns | USP/NF, Ph. Eur | Hanns G. Werner/Signet |
| 5 | VIVAPHARM ® HPMC E3 | Hypromellose (HPMC) | USP/NF, Ph. Eur., JP | JRS PHARMA GmbH + Co. KG |
| 6 | Aerosil ® 200 Pharma | Silicon dioxide | USP/NF, Ph. Eur., JP | Evonik Operations GmbH |
| 7 | EUDRAGIT ® L 30 D-55; T_{gm} 90° C. | Methacrylic Acid and Ethyl | USP/NF, Ph. Eur., JP | Evonik Operations GmbH |
| | | Acrylate Copolymer | | |
| 8 | EUDRAGIT ® NM 30 D; T_{gm} 11° C. | Ethyl Acrylate and Methyl | USP/NF, Ph. Eur., JP | Evonik Operations GmbH |
| | | Methacrylate Copolymer | | |
| 9 | IMWITOR ® 900 K | Glycerol monostearate 40-55%, | USP/NF, Ph. Eur. | Emerson Resources INC/ |
| | | Type II | | Evonik Operations GmbH |
| 10 | TWEEN 80-LQ-(CQ) | Polysorbate 80 | USP/NF, Ph. Eur. | Croda Europe Limited |
| 11 | Magnesium Oxide, Light | Magnesium Oxide | USP | Dead Sea Bromine Company Ltd. |
| 12 | Glycerol anhydrous, EMPROVE ® | Glycerol | USP/NF, Ph. Eur., JP | Merck KGaA |
| 13 | Triethyl Citrate | Triethyl Citrate | USP/NF, Ph. Eur. | Vertellus Holdings LLC |
| 14 | Talc | Talc | USP/NF, Ph. Eur., JP | Imerys S.A. |
| 15 | Sodium hydroxide, EMPROVE ® | Sodium hydroxide | USP/NF, Ph. Eur., JP | Merck KGAA |
| 16 | Vcaps ® Plus capsules, size #3 | Hypromellose Capsules | USP/NF, Ph. Eur., JP | Capsugel France SAS |
| 17 | Vcaps ® Plus capsules, size #0 | Hypromellose Capsules | USP/NF, Ph. Eur., JP | Capsugel France SAS |
| 18 | Vcaps ® Plus capsules, size #00 | Hypromellose Capsules | USP/NF, Ph. Eur., JP | Capsugel France SAS |
| 19 | Klucel ™ EF PHARM | Hydroxypropylcellulose | USP/NF, Ph. Eur., JP | ASHLAND Specialty Ingredients G.P. |

### A. Core Preparation:

### 1. Caffeine Blend:

### 1.1 Composition for Caffeine Blend:

TABLE 2
Composition for Caffeine Capsules for all experiments:

| | Experiment ID | |
|---|---|---|
| | C1 | |
| | Composition | Composition |
| Ingredients | (% w/w) | (mg) # |
| Caffeine anhydrous | 66.70 | 90.0 |
| VIVAPUR ® 101 | 33.30 | 45.0 |
| Total | 100 | 135.0 |

# Note:
Composition of Experiment C1 is also expressed in milligrams for demonstrating percentage alkali on alkali and enteric polymer calculation. Quantities of ingredients in subsequent experiments can be calculated likewise

### 1.2 Process for Caffeine Blend Preparation:

I. Weighed all ingredients in required quantity.
II. Sifted Caffeine and Microcrystalline Cellulose (MCC) PH101 through 40 # sieve.
III. Mixed above material in double cone blender for 15 minutes at 15 RPM.
IV. Blend ready for filling into pre-coated as well un-coated HPMC size 0‘ ’3′ capsules.
V. Blend filled into pre-coated/post-coated HPMC capsules using manual capsule filling machine with change part of size ‘0’/‘3’.

### B. Core Preparation:

### 2. Sotalol Hydrochloride Pellets:

### 2.1 Composition for Sotalol HCl Pellets:

TABLE 3
Composition for Sotalol HCl Pellets for comparative experiments:

| | Composition |
|---|---|
| Ingredients | (% w/w) |
| PHARM-A-SPHERES (MESH 20-25) | 70.92 |
| Sotalol HCl | 21.28 |
| VIVAPHARM ® HPMC E3 | 5.32 |
| Aerosil ® 200 Pharma | 2.48 |
| Water (q.s. to % w/w solids) | q.s. to 27% |
| Total | 100 |

### 2.2 Process for Sotalol Hydrochloride Pellets Preparation:

I. All the ingredients were weighed in required quantity.
II. HPMC [3 cps] was dissolved in water using overhead stirrer, till a clear solution is obtained.
III. Sotalol was sifted through 40 #(400 μm) sieve and mixed together with Aerosil 200 in polybag for 2 min, then this blend was added to the solution of step II.
IV. Suspension was passed through 40 # sieve and used for drug layering on NPS.
V. Used dehumidifier during spraying drug solution on NPS.

TABLE 4
Coating composition for intermediate and enteric coating of comparative and inventive experiments

| | | | | Composition | | |
|---|---|---|---|---|---|---|
| Composition (% w/w) | | | | ( mg/cap)* | Composition (% w/w) | |
| Experiment No. | C1 | C5 | I1 | I1 | C3 | C4 |
| Objective | Post coating with | Post coating with sub | Pre-coating | Pre-coating with | Pre-coating | Coating on core |
| | sub and functional | and functional coating | with sub and | sub and | using 20% | pellets using |
| | coating | | functional | functional | neutralized | neutralized |
| | | | coating | coating | EUDRAGIT ® | EUDRAGIT ® |
| | | | | | L 30 D-55 | L 30 D-55 |
| Core | Vcaps ® Plus | V-Caps plus size #00 | Vcaps ® | Vcaps ® Plus | Vcaps ® Plus | Sotalol |
| | capsules, size #3 | (filled with 700 mg | Plus | capsules, size #3- | capsules, | HCl Pellets |
| | filled according to | VIVAPUR ® | capsules, | capsule empty | size #3 | According to |
| | chapter A | 101/Caffeine | size #3 | weight 48 mg | | chapter B |
| | | anhydrous blend 7:3) | | | | |

Sub coating/Intermediate coating step

| VIVAPHARM ® HPMC E3 | 66.66 | n/a | 66.66 | 2.86 | n/a | n/a |
|---|---|---|---|---|---|---|
| Klucel ™ EF PHARM | n/a | 55.56 | n/a | n/a | | |
| Glycerol anhydrous, | 16.67 | n/a | 16.67 | 0.72 | | |
| EMPROVE ® | | | | | | |
| Magnesium Oxide, Light | 16.67 | 27.78 | 16.67 | 0.72 | | |
| Talc | n/a | 16.67 | n/a | n/a | | |
| Water | q.s.to 10% | q.s.to 10% | q.s.to 10% | q.s.to 10% | | |
| (q.s to % w/w solid) | | | | | | |
| Total | 100 | 100 | 100 | 52.3 | | |
| Polymer weight gain | 0.89 mg/cm² | 5 mg/cm² | 0.89 mg/cm² | | | |
| Total weight gain | 1.3 mg/cm² | 9 mg/cm² | 1.3 mg/cm² | | | |
| Ratio VIVAPHARM ® | 1:0.25 | 1:0.5 | 1:0.25 | | n/a | n/a |
| HPMC E3 or Klucel ™ | | | | | | |
| EF PHARM and | | | | | | |
| Magnesium Oxide, light | | | | | | |

Functional/Enteric coating step

| | | | | | | |
|---|---|---|---|---|---|---|
| EUDRAGIT L 30 D-55 | 66.67 | 58.89 | 67.16 | 9.66 | 67.16 | 60.8 |
| Triethyl Citrate | 6.67 | 11.73 | 7.46 | 1.07 | 7,46 | 6.08 |
| EUDRAGIT NM 30 D | n/a | n/a | 7.46 | 1.07 | 7.46 | |
| IMWITOR ® 900K | n/a | n/a | 14.93 | 2.15 | 14.93 | |
| TWEEN 80-LQ-(CQ) | n/a | n/a | 2.99 | 0.43 | 2.99 | |
| Talc | 33.33 | 29.38 | n/a | n/a | n/a | 30.4 |
| Sodium hydroxide, | n/a | n/a | n/a | n/a | 3.0 | 2.72 |
| EMPROVE ® | | | | | | |
| Water | q.s.to 20% | q.s.to 20% | q.s.to 20% | q.s.to 20% | q.s.to 10% | q.s.to 10% |
| (q.s to % w/w solid) | | | | | | |
| Total weight | 100 | 100 | 100 | 66.68 | 100 | 100 |
| Polymer weight gain | 2.0 mg/cm² | 7.5 mg/cm² | 2.0 mg/cm² | 2.0 mg/cm2 | 1.3 mg/cm2 | 6.1% |
| Total weight gain | 3.0 mg/cm² | 12.8 mg/cm² | 3.0 mg/cm² | 3.0 mg/cm² | 2.0 mg/cm² | 10.0% |

Total Sub coating/Intermediate coating step plus Functional/Enteric coating step

| | | | | | | |
|---|---|---|---|---|---|---|
| Polymer weight gain | 2.89 mg/cm² | 12.5 mg/cm² | 2.89 mg/cm² | 2.89 mg/cm² | 1.3 mg/cm² | 6.1% |
| Total weight gain | 4.3 mg/cm² | 21.8 mg/cm² | 4.3 mg/cm² | 4.3 mg/cm² | 2.0 mg/cm² | 10.0% |

*Note:
Composition of Experiment I1 is also expressed in milligrams for demonstrating percentage alkali on alkali and enteric polymer calculation.
Quantities of ingredients in subsequent experiments can be calculated likewise.
** weight gain (%) calculated based on filled capsule weight.

TABLE 5
Coating composition for intermediate, enteric and top coating of comparative and inventive experiments

| | | | | | | Composition |
|---|---|---|---|---|---|---|
| Composition (% w/w) | | | | | | (mg/cap)* |
| Experiment No. | C2 | I2 | I3 | I4 | I5 | I5 |
| Core | Vcaps ® Plus | Vcaps ® Plus | Vcaps ® Plus | Vcaps ® Plus | Vcaps ® Plus | Vcaps ® Plus |
| | capsules, | capsules, | capsules, | capsules, | capsules, | capsules, size #0, |
| | size #0 | size #0 | size #0 | size #0 | size #0 | empty capsule |
| | | | | | | weight 100 mg |

Sub coating/Intermediate coating step

| VIVAPHARM ® HPMC E3 | n/a | 44.44 | 57.14 | 66.67 | 66.67 | 4.85 |
|---|---|---|---|---|---|---|
| Glycerol anhydrous, | n/a | 11.11 | 14.29 | 16.77 | 16.77 | 1.21 |
| EMPROVE ® | | | | | | |
| Magnesium Oxide, light | n/a | 44.44 | 28.57 | 16.67 | 16.67 | 1.21 |
| Water | n/a | q.s.to 10% | q.s.to 10% | q.s.to 10% | q.s.to 10% | q.s.to 10% |
| (q.s to % w/w solid) | | | | | | |
| Total | n/a | 100 | 100 | 100 | 100 | 107.27 |
| Polymer weight gain | n/a | 0.89 mg/cm² | 0.89 mg/cm² | 0.89 mg/cm² | 0.89 mg/cm² | |
| Total weight gain | n/a | 2.0 mg/cm² | 1.6 mg/cm² | 1.3 mg/cm² | 1.3 mg/cm² | |
| Ratio VIVAPHARM ® HPMC | n/a | 1:1 | 1:0.5 | 1:0.25 | 1:0.25 | |
| E3 and Magnesium Oxide, | | | | | | |
| light | | | | | | |

Functional/Enteric coating step

| | | | | | | |
|---|---|---|---|---|---|---|
| EUDRAGIT ® L30D-55 | 67.16 | 67.16 | 67.16 | 67.16 | 67.16 | 11.35 |
| EUDRAGIT ® NM 30 D | 7.46 | 7.46 | 7.46 | 7.46 | 7.46 | 1.26 |
| IMWITOR ® 900K | 14.93 | 14.93 | 14.93 | 14.93 | 14.93 | 2.52 |
| TWEEN 80-LQ-(CQ) | 2.99 | 2.99 | 2.99 | 2.99 | 2.99 | 0.50 |
| Triethyl Citrate | 7.46 | 7.46 | 7.46 | 7.46 | 7.46 | 1.26 |
| Water | q.s.to 20% | q.s.to 20% | q.s.to 20% | q.s.to 20% | q.s.to 20% | q.s.to 20% |
| (q.s to % w/w solid) | | | | | | |
| Total | 100 | 100 | 100 | 100 | 100 | 124.16 |
| Polymer weight gain | 1.34 mg/cm² | 3.36 mg/cm² | 2.69 mg/cm² | 2.0 mg/cm² | 2.0 mg/cm² | 2.0 mg/cm² |
| Total weight gain | 2.0 mg/cm² | 5.0 mg/cm² | 4.0 mg/cm² | 3.0 mg/cm² | 3.0 mg/cm² | 3.0 mg/cm² |

Top-coat

| | | | | | | |
|---|---|---|---|---|---|---|
| VIVAPHARM ® HPMC E3 | 90.9 | 90.9 | 90.9 | 90.9 | n/a | n/a |
| IMWITOR ® 900K | 5.4 | 5.4 | 5.4 | 5.4 | n/a | n/a |
| TWEEN 80-LQ-(CQ) | 1.1 | 1.1 | 1.1 | 1.1 | n/a | n/a |
| Triethyl Citrate | 2.7 | 2.7 | 2.7 | 2.7 | n/a | n/a |
| Water | 10% w/w | 10% w/w | 10% w/w | 10% w/w | n/a | n/a |
| (q.s to % w/w solid) | | | | | | |
| Total | 100 | 100 | 100 | 100 | n/a | n/a |
| Polymer weight gain | 0.55 mg/cm² | 0.55 mg/cm² | 0.55 mg/cm² | 0.55 mg/cm² | n/a | n/a |
| Total weight gain | 0.6 mg/cm² | 0.6 mg/cm² | 0.6 mg/cm² | 0.6 mg/cm² | n/a | n/a |

Total Sub coating/Intermediate coating step plus Functional/Enteric coating step

| | | | | | | |
|---|---|---|---|---|---|---|
| Polymer weight gain | 1.89 | 4.8 | 4.13 | 3.44 | 2.89 | 2.89 |
| Total weight gain | 2.6 | 7.6 | 6.2 | 4.9 | 4.3 | 4.3 |

TABLE 6
Coating composition for intermediate, enteric and top coating of comparative and inventive experiments
Composition (% w/w)

| | Experiment No. | |
|---|---|---|
| | C6 | C7 |
| Core | Vcaps ® | Vcaps ® |
| | Plus | Plus |
| | capsules, | capsules, |
| | size #0 | size #3 |

Sub coating/Intermediate coating step

| | | |
|---|---|---|
| VIVAPHARM ® HPMC E3 | 44.44 | 66.66 |
| Glycerol anhydrous, EMPROVE ® | 11.11 | 16.67 |
| Magnesium Oxide, light | 44.44 | 16.67 |
| Water (q.s to % w/w solid) | q.s. to 10% | q.s. to 10% |
| Total | 100 | 100 |
| Polymer weight gain | 0.89 mg/cm² | 0.89 mg/cm² |
| Total weight gain | 2.0 mg/cm² | 1.3 mg/cm² |
| Ratio VIVAPHARM ® HPMC E3 and | 1:1 | 1:0.25 |
| Magnesium Oxide, light | | |

Functional/Enteric coating step

| | | |
|---|---|---|
| EUDRAGIT ® L30D-55 | 66.67 | 67.16 |
| EUDRAGIT ® NM 30 D | | 7.46 |
| Triethyl Citrate | 6.67 | 7.46 |
| IMWITOR ® 900 K | n/a | 14.93 |
| TWEEN 80-LQ-(CQ) | n/a | 2.99 |
| Talc | 33.33 | n/a |
| Water (q.s to % w/w solid) | q.s. to 20% | q.s. to 20% |
| Total | 100 | 100 |
| Polymer weight gain | 3.12 mg/cm² | 3.73 mg/cm² |
| Total weight gain | 5.0 mg/cm² | 5.0 mg/cm² |

Top Coat

| | | |
|---|---|---|
| VIVAPHARM ® HPMC E3 | n/a | 90.9 |
| IMWITOR ® 900 K | n/a | 5.4 |
| TWEEN 80-LQ-(CQ) | n/a | 1.1 |
| Triethyl Citrate | n/a | 2.7 |
| Water (q.s to % w/w solid) | n/a | 10% w/w |
| Total | n/a | 100 |
| n/an/aPolymer weight gain | n/a | 0.55 mg/cm² |
| Total weight gain | n/a | 0.6 mg/cm² |

Total Sub coating/Intermediate coating step plus Functional/Enteric coating step

| | | |
|---|---|---|
| Polymer weight gain | 4.01 mg/cm² | 5.17 mg/cm² |
| Total weight gain | 7 mg/cm² | 6.9 mg/cm² |

### 2. Coating Process for Inventive Experiments:

### 2.1 Sub Coating:

### 2.1.1 Sub Coating of Experiment C1, I1 to I5:

I. All ingredients were weighed in required quantity.
II. VIVAPHARM® HPMC E3 was dissolved in water containing Glycerol anhydrous (EMPROVE®) using overhead stirrer, until a clear solution is obtained.
III. Magnesium oxide (light) was added to above solution slowly while stirring and resulted suspension was then allowed to mix for 30 min.
IV. Suspension was passed through 40 # sieve and used for sub coating.

TABLE 7
General Process Parameters for sub coating of comparative
and inventive Experiment C1, I1 to I5:

| General Process Parameters | Experiment C1, |
|---|---|
| for intermediate coating | I1-I5 |
| Equipment | Neocota |

Equipment setup

| | | |
|---|---|---|
| Silicone tube inner diameter | mm | 3.0 |
| Pan size | inch | 14 |
| Number of baffles | No.s | 6 |

Process parameter

| | | |
|---|---|---|
| Pan RPM | RPM | 2-8 |
| Inlet temperature | ° C. | 60-65 |
| Product temperature | ° C. | 30-35 |
| Atomization pressure | bar | 1.0-1.5 |
| Spray rate | g/min | 3-5 |

### 2.1.2 Sub Coating of Experiment C:

I. Add the Klucel™ EF PHARM slowly to half of the water while stirring with a dissolver plate.
II. Keep stirring until a clear solution without unsolved particles results.
III. Add the MgO and the talc to the other half of the water during homogenizing with a Silverson.
IV. Then add this suspension to the polymer solution.
V. The suspension was kept stirring with a magnetic stirrer through the whole coating process

TABLE 8
General Process Parameters for sub coating of
comparative Experiment C5

| General Process Parameters | |
|---|---|
| for intermediate coating | Experiment C5 |
| Equipment | Lodige |
| | Hi-Coater |
| | LHC |
| | 25-35 |

Equipment setup

| | | |
|---|---|---|
| Silicone tube inner diameter | mm | 2.0 |
| Pan size | cm | 30 |
| Number of baffles | No.s | 6 |

Process parameter

| | | |
|---|---|---|
| Pan RPM | RPM | 17 |
| Inlet temperature | ° C. | 35-40 |
| Product temperature | ° C. | 27-30 |
| Atomization pressure | bar | 2.0 |
| Flat pattern air | bar | 1.5 |
| Spray rate | g/min | 3-6.5 |

### 2.2 Enteric Coating:

### 2.2.1 Enteric Coating of Experiment C1, C2, C5, C6 and C7:

I. All the ingredients were weighed in required quantity.
II. TEC and Talc were homogenized in water for 15 minutes then added slowly to the EUDRAGIT® L 30 D-55 dispersion while stirring, resulted suspension was mixed for 30 minutes using overhead stirrer.
III. Suspension was passed through 40 # sieve and used for enteric coating.

### 2.2.2 Enteric Coating of Experiment I1 to I5:

I. All the ingredients were weighed in required quantity.
II. The water is added to the EUDRAGIT® L 30 D-55 while stirring with a conventional stirrer (e.g., propeller stirrer or magnetic stirrer).
III. Preparations of excipient suspension, 40% of the total water was heated up to 70-80° C.
IV. The TWEEN 80-LQ-(CQ) (diluted to 30% aqueous solution), triethyl citrate and IMWITOR® 900 K were homogenized in the heated water using a homogenizer (e. g. Ultra Turrax) for 10 minutes.
V. The remaining 60% of water was stirred into the hot emulsion by using a conventional stirrer and cooled down to room temperature while continuous stirring.
VI. Then the excipient suspension is added to EUDRAGIT® L 30 D-55 while the stirring is continued and stirred for another 10 minutes.
VII. EUDRAGIT® NM 30 D is slowly added to the diluted EUDRAGIT® L 30 D-55 while stirring. Suspension kept stirring.
VIII. After 15 minutes of stirring the suspension is passed over a 40 # sieve. The suspension is stirred over the whole coating process.
IX. Suspension was passed through 40 # sieve and used for enteric coating.

### 2.2.3 Enteric Coating of Experiment C3:

I. All the ingredients were weighed in required quantity.
II. The water is added to the EUDRAGIT® L 30 D-55 while stirring with a conventional stirrer (e.g., propeller stirrer or magnetic stirrer).
III. Sodium Hydroxide dissolved in small amount of water and added to the step II suspension under stirring.
IV. Preparations of excipient suspension, 40% of the total water was heated up to 70-80° C.
V. The TWEEN 80-LQ-(CQ) (diluted to 30% aqueous solution), triethyl citrate and IMWITOR®@900 K were homogenized in the heated water using a homogenizer (e. g., Ultra Turrax) for 10 minutes.
VI. The remaining 60% of water was stirred into the hot emulsion by using a conventional stirrer and cooled down to room temperature while continuous stirring.
VII. Then the excipient suspension is added to EUDRAGIT® L 30 D-55 while the stirring is continued and stirred for another 10 minutes.
VIII. EUDRAGIT® NM 30 D is slowly added to the diluted EUDRAGIT® L 30 D-55 while stirring. Suspension kept stirring.
IX. After 15 minutes of stirring the suspension is passed over a 40 # sieve. The suspension is stirred over the whole coating process.
X. Suspension was passed through 40 # sieve and used for enteric coating

### 2.2.4 Enteric Coating of Experiment C4:

I. All the ingredients were weighed in required quantity.
II. Sodium Hydroxide dissolved in purified water and then added slowly to the EUDRAGIT® L 30 D-55 dispersion while stirring, resulted suspension was mixed for 30 minutes using overhead stirrer.
III. TEC and Talc were homogenized in water for 15 minutes then added slowly to the EUDRAGIT® L 30 D-55 dispersion while stirring, resulted suspension was mixed for 30 minutes using overhead stirrer.
IV. Suspension was passed through 40 # sieve and used for enteric coating.

### 2.2.5 Top Coating of Experiment C2, C7 and I2 to I5:

I. All the ingredients were weighed in required quantity.
II. VIVAPHARM® HPMC E3 was dissolved in water while stirring with a conventional stirrer (e.g., propeller stirrer or magnetic stirrer).
III. Preparations of excipient suspension, 40% of the total water was heated up to 70-80° C.
IV. The TWEEN 80-LQ-(CQ) (diluted to 30% aqueous solution), triethyl citrate and IMWITOR® 900 K were homogenized in the heated water using a homogenizer (e.g., Ultra Turrax) for 10 minutes.
V. The remaining 60% of water was stirred into the hot emulsion by using a conventional stirrer and cooled down to room temperature while continuous stirring.
VI. Then the excipient suspension is added to EUDRAGIT® L 30 D-55 while the stirring is continued.
VII. After 15 minutes of stirring the suspension is passed over a 40 # sieve. The suspension is stirred over the whole coating process.
VIII. Suspension was passed through 40 # sieve and used for enteric coating.

TABLE 9
General Process Parameters for enteric and where
applicable top coating of comparative and inventive
Experiment C1 to C3, C6, C7 and I1 to I5:

| | Experiment |
|---|---|
| General Process Parameters | C1 to C3 and |
| for enteric coating | I1 to I5 |
| Equipment | Neocota |

Equipment setup

| | | |
|---|---|---|
| Silicone tube inner diameter | mm | 3.0 |
| Pan size | inch | 14 |
| Number of baffles | No.s | 6 |

Process parameter

| | | |
|---|---|---|
| Pan RPM | RPM | 2-8 |
| Inlet temperature | ° C. | 45-50 |
| Product temperature | ° C. | 28-32 |
| Atomization pressure | bar | 1.0-1.5 |
| Spray rate | g/min | 3-5 |

### Observation of C3 Experiment During Coating:

There is a processability issue observed when empty capsules coated using neutralized EUDRAGIT® L 30 D-55 due to sticking of capsules during coating. Sticking of capsules observed even at very low spray rate & less solid content of the suspension. Hence process could not complete.

TABLE 10
General Process Parameters for enteric coating
of comparative Experiment C4

| General Process Parameters | |
|---|---|
| for enteric coating | Experiment C4 |
| Equipment used | GPCG 1.1 |
| | bottom spray |

Equipment setup

| | | |
|---|---|---|
| Silicone tube inner diameter | mm | 3.0 |
| Air distribution plat | — | B |
| Column height | mm | 20 |
| Nozzle bore | mm | 0.8 |

Process data

| | | |
|---|---|---|
| Filter shaking mode | — | Asynchronous |
| Filter shaking | sec | 5 |
| Filter shaking pause | sec | 100 |
| Air flow mode | — | Auto |
| Air flow | CFM | 50-75 |
| Atomization pressure | bar | 1.0-1.1 |
| Inlet temperature | ° C. | 25-35 |
| Product temperature | ° C. | 20-25 |
| Spray rate | g/min | 2-8 |

TABLE 11
General Process Parameters for enteric coating
of comparative Experiment C5

| General Process Parameters | |
|---|---|
| for intermediate coating | Experiment C5 |
| Equipment | Lodige |
| | Hi-Coater |
| | LHC |
| | 25-35 |

Equipment setup

| | | |
|---|---|---|
| Silicone tube inner diameter | mm | 2.0 |
| Pan size | cm | 30 |
| Number of baffles | No.s | 6 |

Process parameter

| | | |
|---|---|---|
| Pan RPM | RPM | 17 |
| Inlet temperature | ° C. | 35-40 |
| Product temperature | ° C. | 27-30 |
| Atomization pressure | bar | 2.0 |
| Flat pattern air | bar | 1.5 |
| Spray rate | g/min | 3-6.5 |

TABLE 12
Performance of comparative and inventive experiments:

| Experiment No. | | |
|---|---|---|
| C1 | I1 | C5 |

Sub coat/Intermediate coat

| | | | |
|---|---|---|---|
| Polymer weight gain | 0.89 mg/cm² | 0.89 mg/cm² | 5 mg/cm² |
| Total weight gain | 1.3 mg/cm² | 1.3 mg/cm² | 9 mg/cm² |

Functional/Enteric coat

| | | | | | |
|---|---|---|---|---|---|
| Polymer weight gain | 2 mg/cm² | | 2 mg/cm² | | 7.5 mg/cm² |
| Total weight gain | 3.0 mg/cm² | 4.0 mg/cm² | 3.0 mg/cm² | 4.0 mg/cm² | 12.8 mg/cm² |

Dissolution testing in acid stage media

| | | | | | |
|---|---|---|---|---|---|
| pH 1.2 (120 min) | Fail | Pass | Pass | Pass | Fail |

Dissolution testing (% drug release) in buffer stage media

| | | | | | |
|---|---|---|---|---|---|
| pH 5.0 (45 min) | n/a | 32.8 | 56.9 | 89.5 | n/a |
| pH 5.0 (60 min) | n/a | 39.3 | 80.3 | 94.9 | n/a |
| pH 4.5 (45 min) | n/a | n/a | n/a | n/a | n/a |
| pH 4.5 (60 min) | n/a | n/a | n/a | n/a | n/a |
| pH 3.0 (45 min) | n/a | n/a | 70.3 | 44.9 | n/a |
| pH 3.0 (60 min) | n/a | n/a | 97.4 | 69.9 | n/a |

TABLE 13
Performance of comparative and inventive experiments:

| | | | | | | |
|---|---|---|---|---|---|---|
| Experiment No. | C2 | I2 | I3 | I4 | I5 | C4 |

Sub coat/Intermediate coat

| | | | | | | |
|---|---|---|---|---|---|---|
| Polymer weight gain | n/a | 0.89 mg/cm² | 0.89 mg/cm² | 0.89 mg/cm² | 0.89 mg/cm² | n/a |
| Total weight gain | n/a | 2.0 mg/cm² | 1.6 mg/cm² | 1.3 mg/cm² | 1.3 mg/cm² | n/a |

Functional/Enteric coat

| | | | | | | |
|---|---|---|---|---|---|---|
| Polymer weight gain | 1.34 mg/cm² | 3.36 mg/cm² | 2.69 mg/cm² | 2.0 mg/cm² | 2.0 mg/cm² | 6.1% |
| Total weight gain | 2.0 mg/cm² | 5.0 mg/cm² | 4.0 mg/cm² | 3.0 mg/cm² | 3.0 mg/cm² | 10.0% |

Top coat

| | | | | | | |
|---|---|---|---|---|---|---|
| Polymer weight gain | 0.55 mg/cm² | 0.55 mg/cm² | 0.55 mg/cm² | 0.55 mg/cm² | NA | NA |
| Total weight gain | 0.6 mg/cm² | 0.6 mg/cm² | 0.6 mg/cm² | 0.6 mg/cm² | NA | NA |

Dissolution testing in acid stage media

| | | | | | | |
|---|---|---|---|---|---|---|
| pH 1.2 (120 min) | Pass | Pass | Pass | Pass | Pass | Pass |

Dissolution testing (% drug release) in buffer stage media

| | | | | | | |
|---|---|---|---|---|---|---|
| pH 5.0 (45 min) | Capsules were | 100.0 | 102.2 | 67.7 | 91.6 | 102.0 |
| pH 5.0 (60 min) | found intact after 2 | 102.0 | 104.8 | 72.9 | 95.1 | 101.6 |
| pH 4.5 (45 min) | hours and no drug | n/a | n/a | n/a | n/a | 6.9 |
| pH 4.5 (60 min) | release observed. | n/a | n/a | n/a | n/a | 21.9 |
| pH 3.0 (45 min) | | 66.1 | 94.3 | 16.7 | 11.0 | 6.4 |
| pH 3.0 (60 min) | | 71.7 | 101.1 | 18.6 | 15.0 | 11.6 |

TABLE 14
Performance of comparative and inventive experiments:

| Experiment No. | |
|---|---|
| C6 | C7 |

Sub coat/Intermediate coat

| | | | | |
|---|---|---|---|---|
| Polymer weight gain | 0.89 mg/cm² | 0.89 mg/cm² | 0.89 mg/cm² | 0.89 mg/cm² |
| Total weight gain | 2.0 mg/cm² | 1.3 mg/cm² | 1.6 mg/cm² | 1.3 mg/cm² |

Functional/Enteric coat

| | | | | |
|---|---|---|---|---|
| Polymer weight gain | 3.12 mg/cm² | 2.24 mg/cm² | 2.99 mg/cm² | 3.73 mg/cm² |
| Total weight gain | 5.0 mg/cm² | 3.0 mg/cm² | 4.0 mg/cm² | 5.0 mg/cm² |

Top coat

| | | | | |
|---|---|---|---|---|
| Polymer weight gain | n/a | 0.55 mg/cm² | 0.55 mg/cm² | 0.55 mg/cm² |
| Total weight gain | n/a | 0.6 mg/cm² | 0.6 mg/cm² | 0.6 mg/cm² |

Dissolution testing in acid stage media

| | | | | |
|---|---|---|---|---|
| pH 1.2 (120 min) | Pass | Failed | Pass | Pass |

Dissolution testing (% drug release) in buffer stage media

| | | | | |
|---|---|---|---|---|
| pH 5.0 (45 min) | 30.0 | n/a | 32.8 | 30.0 |
| pH 5.0 (60 min) | 46.5 | n/a | 39.3 | 34.4 |
| pH 4.5 (45 min) | No drug release | n/a | n/a | n/a |
| pH 4.5 (60 min) | detected | n/a | n/a | n/a |
| pH 3.0 (45 min) | | n/a | n/a | n/a |
| pH 3.0 (60 min) | | n/a | n/a | n/a |
| Observation | Squeezing of | | | |
| | capsules; Cracks | | | |
| | observed on | | | |
| | capsule surface | | | |

### SEM Method Description:

Pre and post coated capsules were tested for thickness of coating layers thickness using SEM analysis.

### Equipment:

Make: Thermo Fisher Scientific
Model: FEI Quanta 200, Netherland

### Sample Preparation:

Samples are to be randomly taken from the batch of coated dose units i.e., capsules. The taken samples were cut with a sharp-edged cutter, to get cross section of coated capsules at room temperature. The cross section of sample was fixed in an upright position on the sample mounting disc and analyzed for thickness determination.

### Equipment Set Up:

Analysis method: Samples are analyzed under Scanning Electron Microscope in Low Vacuum mode with pressure 65 pascals at different magnification.
Voltage (HV) was kept at 20 KV
Detector used was Large Field Detector (LFD).

TABLE 15
Coating thickness evaluation of comparative and
inventive experiments by SEM study:

| Experiment No. | C1 | I1 | C2 | I2 | I3 | I4 | I5 |
|---|---|---|---|---|---|---|---|
| Intermediate coating | 8.7 | 10 | NA | | 13.0 | 8.8 | 6.65 |
| thickness (μm) | | | | | | | |
| Enteric coating | 21.33 | 27.66 | 25.83 | 36.6 | 28.0 | 21.47 | 18.62 |
| thickness (μm) | | | | | | | |
| Top layer coating | NA | NA | 8.5 | | 10.0 | 8.7 | NA |
| thickness (μm) | | | | | | | |

1. A process for preparing a polymer-coated hard shell capsule comprising at least an intermediate coating layer, an enteric coating layer, and optionally a top coating layer, suitable as a container for pharmaceutical or nutraceutical biologically active ingredients, and further comprising a body and a cap, wherein in a closed state, the cap overlaps the body either in a pre-locked state or in a final-locked state, the process comprising:
providing the hard shell capsule in the pre-locked state and
coating the hard shell capsule with a first coating solution, suspension or dispersion comprising or consisting of
a1) at least one polymer;
b1) at least one alkaline agent;
c1) optionally at least one glidant; and
d1) optionally at least one additive, different from a1) to c1);
to obtain the intermediate coating layer of the hard shell capsule in the pre-locked state; and thereafter coating the hard shell capule with a second coating solution, suspension or dispersion, which is different from the first coating solution, suspension or dispersion, comprising or consisting of
a2) at least two polymers;
b2) optionally at least one glidant;
c2) optionally at least one emulsifier;
d2) optionally at least one plasticizer;
e2) optionally at least one biologically active ingredient; and
f2) optionally at least one additive, different from a2) to e2);
wherein a first polymer of the at least two polymers a2) is an anionic polymer having a T_{gm}≥35° C.;
wherein a second polymer of the at least two polymers a2) is a polymer having a T_{gm} of ≤30° C.:
to obtain the enteric coating layer of the hard shell capsule in the pre-locked state, wherein a total coating amount of the coating layers is 2.0 to 10 mg/cm².

2. The process according to claim 1, wherein a base material of the body and the cap is at least one selected from the group consisting of hydroxypropyl methyl cellulose, starch, gelatin, pullulan, a copolymer of a C₁-C₄-alkylester of (meth)acrylic acid and (meth)acrylic acid, and mixtures thereof.

3. The process according to claim 1, wherein the at least one polymer a1) and/or the at least two polymers a2) is/are at least one selected from the group consisting of (meth)acrylate copolymer, hydroxypropyl methylcellulose (HPMC), hydroxypropyl cellulose (HPC), and mixtures thereof.

4. The process according to claim 1, wherein the anionic polymer a2) is
i) a Core-Shell polymer, which is a copolymer obtained by a two stage emulsion polymerization process with a core with 70 to 80% by weight, comprising polymerized units of 65 to 75% by weight of ethyl acrylate and 25 to 35% by weight of methyl methacrylate, and a shell with 20 to 30% by weight, comprising polymerized units of 45 to 55% by weight ethyl acrylate and 45 to 55% by weight methacrylic acid; or
ii) an anionic polymer obtained by polymerizing 25 to 95% by weight C₁-C₁₂-alkyl esters of acrylic acid or of methacrylic acid and 75 to 5% by weight (meth)acrylate monomers with an anionic group; or
iii) a (meth)acrylate copolymer obtained by polymerizing methacrylic acid and ethyl acrylate, methacrylic acid and methyl methacrylate, ethyl acrylate and methyl methacrylate or methacrylic acid, methyl acrylate and methyl methacrylate: or iv) a (meth)acrylate copolymer obtained by polymerizing 40 to 60% by weight of methacrylic acid and 60 to 40% by weight of ethyl acrylate; or
v) a (meth)acrylate copolymer obtained by polymerizing 5 to 15% by weight methacrylic acid, 60 to 70% by weight of methyl acrylate and 20 to 30% by weight methyl methacrylate; or mixtures thereof.

5. The process according to claim 1, wherein the second polymer of a2) is
i) a (meth)acrylate copolymer obtained by polymerizing at least two C₁-C₄-alkyl acrylate monomers and 0 to less than 5% by weight of methacrylic acid or acrylic acid: or
ii) a (meth)acrylate copolymer obtained by polymerizing 20 to 40% by weight of ethyl acrylate, 60 to 80% by weight of methyl methacrylate and 0 to less than 5% by weight of methacrylic acid or acrylic acid; or
iii) a (meth)acrylate copolymer obtained by polymerizing 60 to 80% of ethyl acrylate and 40 to 20% by weight of methyl methacrylate.

6. The process according to claim 1, wherein two of the at least two polymers a2) are a (meth)acrylate copolymer obtained by copolymerizing 40 to 60% by weight of methacrylic acid and 40 to 60% by weight of ethyl acrylate; and a (meth)acrylate copolymer obtained by polymerizing 60 to 78% of ethyl acrylate and 20 to 38% by weight of methyl methacrylate and optionally up to 2% by weight of (meth)acrylic acid.

7. The process according to claim 1, wherein the at least one polymer a1)
i) is selected from the group consisting of hydroxyethyl cellulose (HEC), hydroxypropyl cellulose (HPC), hydroxypropyl methyl cellulose (HPMC), hydroxyethyl methyl cellulose (HEMC), ethyl cellulose (EC), methyl cellulose (MC), cellulose esters, cellulose glycolates, polyethylene glycols, polyethylene oxides, polyvinyl pyrrolidone, polyvinyl acetate, polyvinyl alcohol, and a mixture thereof; and/or
ii) is present in 5 to 90% by weight, based on 4 total weight of the intermediate layer.

8. The process according to claim 1, wherein the at least one alkaline agent b1)
i) is selected from the group consisting of calcium oxide, calcium carbonate, magnesium carbonate, magnesium oxide, sodium carbonate, sodium bicarbonate, sodium hydroxide and any mixtures thereof; and/or
ii) is present in 10 to 75% by weight, based on a total weight of the intermediate layer.

9. The process according to claim 1, wherein in the first coating solution, the at least one polymer to the at least one alkaline agent is present in a weight ratio of 1:1 to 1:0.25.

10. The process according to claim 1, wherein at least one glidant is present in the first and/or second coating solution, suspension or dispersion.

11. The process according to claim 1, wherein at least one emulsifier is present in the second coating solution, suspension or dispersion.

12. The process according to claim 1, wherein at least one plasticizer is present in the second coating solution, suspension or dispersion.

13. The process according to claim 1, wherein up to 400% by weight, based on a total weight of the at least one polymer, of at least one additive are comprised in the first and/or second coating solution, suspension or dispersion.

14. The polymer-coated hard shell capsule, obtained from the process according to claim 1.

15. The polymer-coated hard shell capsule according to claim 14, wherein the polymer-coated hard shell capsule provides less than 10% drug release at 0.1N HCl for 120 minutes followed by at least 80% drug release at pH value 5 within additional 150 min.
